(19) United States
(12) Patent Application Publication
(10) Pub. No.: US 2014/0273033 A1
(43) Pub. Date: Sep. 18, 2014

(54) BIOMARKERS FOR MULTIPLE SCLEROSIS

(71) Applicant: Universiteit Hasselt, Diepenbeek (BE)

(72) Inventors: Veerle Somers, Sint-Truiden (BE); Pieter Stinissen, Diepenbeek (BE)

(73) Assignee: Universiteit Hasselt, Diepenbeek (BE)

(21) Appl. No.: 14/290,782

(22) Filed: May 29, 2014

Related U.S. Application Data
(63) Continuation-in-part of application No. 12/595,597, filed on Apr. 14, 2010, now Pat. No. 8,772,225.

(30) Foreign Application Priority Data
Apr. 12, 2007 (EP) 07106081.8

(51) Int. Cl. G01N 33/564; C07K 14/47

(52) U.S. Cl. CPC G01N 33/564; C07K 14/4713
USPC 435/7.92; 436/501; 530/350

(57) ABSTRACT

Described is the diagnosis of neurological disorders, more specifically, the diagnosis of multiple sclerosis. A biomarker panel is provided that can be used to detect if a subject has multiple sclerosis. Also described are methods of identification of such biomarkers.

### CROSS-REFERENCE TO RELATED APPLICATIONS

This application is a continuation-in-part of co-pending U.S. patent application Ser. No. 12/595,597, filed Apr. 14, 2010, U.S. Pat. No. ______, which application is a national phase entry under 35 U.S.C. §371 of International Patent Application PCT/EP2008/054479, filed Apr. 14, 2008, designating the United States of America and published in English as International Patent Publication WO 2008/125651 A3 on Oct. 23, 2008, which claims the benefit under Article 8 of the Patent Cooperation Treaty and under 35 U.S.C. §119(e) to European Patent Application Serial No. 07106081.8, filed Apr. 12, 2007, the disclosure of each of which is hereby incorporated herein in its entirety by this reference.

### TECHNICAL FIELD

The disclosure relates to medicine, biotechnology, and the diagnosis of neurological disorders, more specifically, to the diagnosis of multiple sclerosis. A biomarker panel is provided that can be used to detect if a subject has multiple sclerosis. Also described are methods of identification of such biomarkers.

### BACKGROUND

Multiple sclerosis (MS) affects more than 350,000 people in the U.S. and 2.5 million worldwide. In the U.S., prevalence estimates vary between 5 and 119 per 100,000 and healthcare costs are estimated to be more than $10 billion annually in the U.S. alone. It is the most common neurological disease in young adults, with the risk of subsequent chronic functional impairment and disability after 10-15% of disease duration. The disease is characterized initially in 80-90% of patients by recurrent neurological events (relapses) that are attributable to multifocal lesions within the CNS. Further disease courses vary from benign to classical relapsing-remitting (RR), primary (PP) and secondary (SP) chronic progressive or rare fulminant disease course. MS is considered to be of autoimmune origin and is characterized neuropathologically by variable extents of focal inflammation, demyelination, axonal damage, gliotic scarring and atrophy, but also by remyelination and regeneration in the CNS. This has led, together with the clinical variability, to the concept of MS as a heterogeneous disease with respect to four pathogenetic mechanisms of demyelination.^{1, 2} One of these pathogenetic subtypes is characterized neuropathologically by antibody-dependent immune mechanisms involved in the formation of MS lesions.^{1, 3}

During the past years, an important role of autoreactive B cells and autoantibodies has been demonstrated.⁴ Recent studies uniformly showed clonal expansion of antibody-secreting B cells in the CNS and cerebrospinal fluid (CSF) of patients with MS.^{5, 6}Furthermore, detection of oligoclonal antibodies in CSF of patients with neurological diseases has been associated with the presence of MS. Numerous studies have reported the recognition of central nervous system (CNS) myelin autoantigens such as myelin basic protein (MBP), proteolipid lipoprotein, myelin oligodendrocyte glycoprotein, myelin associated glycoprotein by autoantibodies present in CSF and serum of MS patients, but also in patients with other-inflammatory neurological diseases (OIND) and non-inflammatory neurological diseases (NIND) as well as healthy controls.⁷⁻¹¹

A physician may diagnose MS in some patients soon after the onset of the illness. In others, however, doctors may not be able to readily identify the cause of the symptoms, leading to years of uncertainty and multiple diagnoses. The vast majority of patients are mildly affected, but in the worst cases, MS can render a person unable to write, speak or walk. Unfortunately, no single laboratory test is yet available to prove or rule out MS. Therefore, there is a great need in the art for improved diagnostic tests for MS. The development of a panel of biomarkers, specific for different pathophysiological mechanisms, will be crucial for the further understanding of the pathogenesis of MS, as well as diagnosis, classification, disease activity, and theranostic applications.

In the disclosure, we report the identification of autoantibody binding peptides/proteins that are highly specific for MS patients. The results obtained were also correlated to disease duration, disability and different clinical course of disease. The autoantibody profiles against these selected peptides can be used as a biomarker panel for the specific detection of MS. In addition to the identification of these novel autoantibody targets, the role of one particular autoantibody target, SPAG16—a protein with unknown function in MS and in the brain, in MS pathogenesis was elucidated. SPAG16 was identified as an autoantibody target in MS with pathogenic relevance. Passive transfer experiments with anti-SPAG16 antibodies in experimental autoimmune encephalomyelitis (EAE), an animal model of MS, demonstrated that increased SPAG16 levels correlate with increased disease severity and worse prognosis.

### SUMMARY OF THE DISCLOSURE

Described, among other things, are methods of detecting at least one specific antibody to Sperm-Associated Antigen 16's (SPAG16's) presence and/or quantity. Such a method comprises contacting a body fluid (e.g., cerebrospinal fluid, blood, blood serum, and/or blood plasma) from a mammal (e.g., a human) with SPAG16 protein and/or a peptide fragment of at least 5 consecutive amino acids of SPAG16, and detecting (e.g., via an immune-enzymatic process comprising an enzyme-linked immunosorbant assay (ELISA), an immunofluorescent technique, a radioimmunological assay (RIA), immunoblotting, and/or a LINE blot) the presence or measuring the quantity of specific antibodies bound to SPAG16 protein and/or the peptide fragment. In such a method, the method utilizes a peptide fragment of SPAG16 corresponding to SEQ ID NO:8. The method may further comprises detecting the presence or quantity of at least one antibody that has a specificity for a peptide comprising a peptide selected from the group consisting of SEQ ID NO:1, SEQ ID NO:2, SEQ ID NO:3, SEQ ID NO:4, SEQ ID NO:5, SEQ ID NO:6, SEQ ID NO:7, and a fragment comprising at least 5 consecutive amino acids of SEQ ID NO:1, SEQ ID NO:2, SEQ ID NO:3, SEQ ID NO:4, SEQ ID NO:5, SEQ ID NO:6, or SEQ ID NO:7.

Also described are methods of diagnosing multiple sclerosis in a mammal, the method comprising: contacting a body fluid of the mammal with Sperm-Associated Antigen 16 (SPAG16) protein or a peptide fragment of at least 5 consecutive amino acids of SPAG16 protein, and detecting the presence or measuring the quantity of at least one specific antibody bound to SPAG 16 protein or the peptide fragment, wherein the presence of and/or increased quantity of bound antibody as compared to a negative control indicates multiple sclerosis in the mammal.

Further described are methods for evaluating the prognosis and/or disease severity of multiple sclerosis in a subject suffering therefrom, such a method comprising contacting a body fluid of the subject with Sperm-Associated Antigen 16 (SPAG16) protein and/or a peptide fragment of at least 5 consecutive amino acids of SPAG16 protein, and detecting the presence or measuring the quantity of at least one specific antibody bound to SPAG16 protein and/or the peptide fragment, wherein the decreased or increased concentration of the at least one specific antibody indicates the prognosis of multiple sclerosis in the subject.

Further described is a method for selecting a patient for a specific therapeutic treatment of multiple sclerosis or evaluating the therapeutic treatment of multiple sclerosis in a subject suffering therefrom, the method comprising: contacting a body fluid of the subject with Sperm-Associated Antigen 16 (SPAG16) protein or a peptide fragment of at least 5 consecutive amino acids of SPAG16 protein, and detecting the presence or measuring the quantity of the specific antibodies bound to SPAG16 protein or the peptide fragment, wherein the presence or quantity of the antibody leads to an election of a specific therapeutic treatment of multiple sclerosis in the subject.

Described is a diagnostic kit comprising Sperm-Associated Antigen 16 (SPAG16) protein or a peptide fragment of at least 5 consecutive amino acids of SPAG16 protein, reagents for making a medium appropriate for an immunological reaction to occur, and reagents that detect an antigen/antibody complex that has been produced by the immunological reaction. The kit comprises an antibody specific for SPAG16 and/or a peptide fragment of at least 5 consecutive amino acids of SPAG16 protein.

Further described is a SPAG16 or a peptide fragment of at least 5 consecutive amino acids of SPAG16 protein bound to a solid support. Included is a peptide comprising SEQ ID NO:8 bound to a solid support or label.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Expression profile of novel antigenic targets in normal tissues. Expression patterns are shown for UH-CSF1.4 and UH-CSFP1.7. The lower panel shows a control hybridization with an actin probe. Lane 1: brain; lane 2: heart; lane 3: skeletal muscle; lane 4: colon; lane 5: thymus; lane 6: spleen; lane 7: kidney; lane 8: liver; lane 9: small intestine; lane 10: placenta; lane 11: lung; lane 12: peripheral blood lymphocytes.

FIG. 2: UH-CSFP1.7 and UH-CSFP1.8 protein expression in E. coli. UH-CSFP1.7 was cloned as antigen (6.1 kDa) and the UH-CSFP1.8 protein was cloned as partial*(13.3 kDa) and full-length protein (20.3 kDa) with a 16.7 kDa thioredoxin fusion (His tag) resulting in 22.8 kDa protein for UH-CSFP1.7 and 30 kDa partial and 37 kDa full length band for UH-CSFP1.8 (SPAG16 protein) on SDS-PAGE after Coommassie staining.

FIG. 3: SAS procedure. a) A phage-displayed MS cDNA repertoire is preincubated with MS patient CSF antibodies. (b) MS-specific antigens displayed on phage (black) bind to MS-antigen specific patient IgG (black). (c) Phage antigen-IgG complexes (black) are captured on a surface coated with polyclonal anti-human IgG (checked). (d) Nonrelevant phages are washed away, and CSF-IgG specific phages are eluted. (e) Selected phages are used for reinfection of bacteria. (f) Selected phages are amplified and used for further rounds of selection.

FIG. 4: Solution phase assays demonstrate high affinity and specificity of CSF antibodies to UH-CSFP1.1. The UH-CSFP1.1 peptide was pre-incubated at different dilutions with MS-CSF8 and MS-CSF26, respectively, and subsequently, the remaining immunoreactivity measured by ELISA. Competition by the UH-CSFP1.1 peptide is displayed. No competition was measured with the random peptide.

FIG. 5: Histogram showing reactivity against UH-CSFP1.1 peptide of ten random clones tested by ELISA assay. A positive signal was obtained for clone 7.

FIG. 6: Protein expression of UH-CSFP1.3 and UH-CSFP1.6 (partial).

FIG. 7: Reactivity of eight individual CSF samples against UH-CSFP1.1, UH-CSFP1.2, UH-CSFP1.4, UH-CSFP1.5 and negative control.

FIG. 8: Antibody reactivity towards UH-CSFP1.6 in serum from 16 randomly selected MS patients, 15 NIND/OIND patients and 16 healthy controls. The horizontal line represents the cut-off value.

FIG. 9: Human SPAG16 isoforms, purity of the recombinant protein and specificity of in-house produced monoclonal anti-SPAG16 antibodies. (Panel A) Different human SPAG16 isoforms in the NCBI database. Isoform 1 and 2 are experimentally validated (black); other putative (*) isoforms are depicted in white. The initially identified autoantibody reactive region of 121 aa using serological antigen selection (SAS) is marked to indicate that the autoreactive antibodies could bind multiple isoforms of the protein. (Panel B) The purity of the produced recombinant proteins was verified by Coomassie Brilliant Blue staining. M: protein marker; S: recombinant SPAG16 (˜37 kDa; with thioredoxin); T: recombinant thioredoxin. (Panel C) Western blot analysis of our in-house produced mouse monoclonal antibodies against SPAG16 showed binding to both human isoform 1 (˜71 kDa) and 2 (˜25 kDa) as confirmed in a testis lysate.

FIG. 10: SPAG16 is an autoantibody target in MS patients. (Panel A) IEF was used to separate the CSF IgG of four MS patients with different MS subtypes. After separation, affinity-mediated immunoblotting was performed using uncoated (TOTAL; showing total IgG), SPAG16-coated (SPAG) or the control protein thioredoxin-coated (THIO) membranes. SPAG16-specific oligoclonal bands in the CSF are marked with arrows in patient 161 (PP-MS) and 230 (SP-MS). Patient 233 (RR-MS) and 270 (SP-MS) did not display these specific bands. (Panel B) Anti-SPAG16 antibody levels in plasma were analyzed with recombinant protein ELISA. Background plasma reactivity was measured against recombinant THIO. The cut-off was determined by ROC-analysis (dashed line; P=0.00018). Anti-SPAG16 antibody levels were compared with the Kruskal-Wallis test of One-Way ANOVA with an overall P<0.01. P-values of post-tests between two groups (Dunn's multiple comparison test) are depicted in the graph. (Panel C) Analysis of the isotype of the anti-SPAG16 antibodies in 22 seropositive MS patients.

FIG. 11: Murine SPAG16 isoforms, binding of in-house produced monoclonal anti-SPAG16 antibodies (mAbs) to murine spinal cord and competition ELISA with antibody positive MS plasma. (Panel A) Validated murine SPAG16 isoforms in the NCBI database. Isoform 1 is also referred to as SPAG16L (long), while isoform 2 is also referred to as SPAG16S (short). The initially identified autoantibody reactive region of 121 aa using serological antigen selection (SAS) is marked to indicate that the autoreactive antibodies could bind multiple isoforms of the protein due to sequence homology. (Panel B) Lane 1: These mAbs also bind a murine SPAG16 protein of ˜47 kDa in mouse spinal cord, corresponding to a ˜405 aa protein (SPAG16 isoform 3) found on NCBI (NP_{—}001258462.1). Blocking with human recombinant SPAG16 results in the disappearance of the band, indicating its specificity (Lane 2) and the band is not visible when incubating with an isotype control antibody (Lane 3). (Panel C) To confirm the specificity of the produced mAbs, supernatant of four antibody producing hybridoma cell lines (5F10, 1F1, 3H5 and an isotype control) was added to antibody-positive MS plasma in a standard protein ELISA to allow competition of binding to coated SPAG 16. Competition between the added mAb and the antibody-positive MS plasma resulted in a dose-dependent decrease in measured OD signals compared to the OD obtained by the antibody-positive MS plasma without added monoclonal antibody, indicating competition between MS Abs and monoclonal mouse Abs. These results confirmed that the produced mAbs were representative for the detected MS plasma anti-SPAG16 immunoreactivity.

FIG. 12: Anti-SPAG16 antibodies exacerbate EAE. (Panel A) EAE score (overall disease course: repeated measures ANOVA, P<0.01) and (Panel B) daily weights of mice receiving anti-SPAG16 (--▪--, n=5) or isotype control (-▴-, n=4) mAbs. Representative data from one experiment are shown; n=15 for anti-SPAG16 antibody-treated group and n=16 for isotype control treated group for two replicate studies giving equivalent results. Data are expressed as mean±SEM. *P<0.05 according to Mann-Whitney U testing. (Panel C) F4/80 staining in the spinal cord in mice treated with anti-SPAG16 mAbs (Panel D) or isotype control mAbs. Representative pictures are shown per group. Quantitative (fluorescent) IHC analysis was performed; (Panel E) numbers of F4/80 positive microglia/macrophages and (Panel F) CD3+ T cells are shown on day 17 post passive antibody transfer. (Panel G) The extent of demyelination is given by the percentage of MBP loss. (Panel H) The % of GFAP+ astrocytes in the spinal cord is shown. Immunoglobulin deposition in the spinal cord of animals receiving anti-SPAG16 or control mAbs is shown as the percentage of the (Panel I) IgM or (Panel J) IgG positive area. Data were compared with Mann-Whitney test. *, P<0.05. Scale bars: (C-D) 100 μm.

FIG. 13: SPAG16 is up-regulated in astrocytes within MS brain lesions and EAE spinal cord lesions. Representative IHC image of (Panel A) SPAG16 and (Panel B) isotype control staining in normal white matter (NWM) from control brain tissue (n=5). (Panel C) SPAG16 staining in grey matter from human control brain tissue. Stained neuron is enlarged in top right panel. (Panel D) Double-staining of SPAG16 (green) and neuronal nuclei (NeuN; red) in grey matter from control brain. Single color images are shown at the right. (Panel E) Delineation of an active MS lesion using MBP staining and (Panel F) HLA-DR/DP/DQ (MHCII) staining to indicate inflammatory cells. (Panel G) Representative SPAG16 staining in delineated MS lesion (brain material from seven MS patients was analyzed). (Panel H) Double-staining of SPAG16 (green) and an astrocyte marker glial fibrillary acidic protein (GFAP; red) in a MS lesion. (Panel I) 400× magnification of the SPAG16 stained MS lesion in Panel G. (Panel J) SPAG16 staining in astrocytes in MS lesion. (Panel K) Representative SPAG16 staining in the spinal cord of a healthy mouse (n=4). (Panel L) Immunofluorescence image of SPAG16 expression (green) in astrocytes in EAE spinal cord (n=5). (Panel M) SPAG16 staining at 40× magnification in EAE spinal cord. (Panel N) Macrophage (F4/80) staining indicates the EAE spinal cord infiltrations. (Panel O) SPAG16 staining at 400× magnification in the delineated region in EAE spinal cord in Panel (M). (Panel P) Fluorescent double-staining for SPAG16 (green) and GFAP (red) in EAE spinal cord. Scale bars: (Panels E-F, M) 200 μm; (Panels G, K) 100 μm; (Panels C-D, L, N-P) 50 μm; (Panels A-B, H-I) 20 μm; (Panel J) 10 μM.

### DETAILED DESCRIPTION

Identified are a set of biomarkers that can be used for the detection of Multiple Sclerosis (MS) in patients. Biomarkers were isolated with the technology of Serological Antigen Selection (SAS) wherein antigens (i.e., biomarkers) were identified that bind to antibodies present in cerebrospinal fluid (CSF) in patients suffering from Multiple Sclerosis. More specifically, a cDNA phage display library comprising cDNA products derived from MS brain plaques—expressed as a fusion to minor coat protein pVI of filamentous phage M13—was panned to identify cDNA clones that bind auto-antibodies in CSF specimens from MS patients. A biomarker panel of eight antigenic cDNA targets that showed 86% specificity and 45% sensitivity in discriminating MS patients and controls was retrieved. Besides a role in the immediate (early) diagnosis of patients suspected for MS, the biomarker panel (i.e., the antigenic cDNA targets) can be used to assist in sub-typing MS patients.

One of the eight autoantibody targets identified is Sperm-Associated Antigen 16 isoform 2 (SPAG16-2). The SPAG16 gene encodes two major transcripts. SPAG16-2 is the smaller transcript that codes for the 20.4-kDa isoform, whilst the other transcript encodes for the 71-kDa SPAG16 isoform 1 (SPAG16-1). Other putative isoforms have also been identified. SPAG16-1 is part of the axoneme (microtubular backbone) in motile cilia and in sperm cells and plays a role in motility. The exact function of SPAG16-2 is unknown.^{36, 37} Though most research regarding the function of SPAG16 has focused on its role in male fertility, SPAG16 (either isoform) expression is not restricted to sperm cells. Recent data reveal its presence in many tissues, including other tissues with motile cilia such as the lungs and brain ventricles but also in hematopoietic cells in the bone marrow, fibroblast-like synoviocytes in rheumatoid arthritis and in certain cancers.³⁷⁻⁴⁰

The role of SPAG16—a protein with unknown function in MS and in the brain—was further investigated in MS pathogenesis. SPAG16 was validated as a target of the humoral autoimmune response in both MS patients' CSF and plasma. Next, we used passive antibody transfer of anti-SPAG16 antibodies in experimental autoimmune encephalomyelitis (EAE), an animal model of MS, to demonstrate the pathogenic relevance of these antibodies in vivo. SPAG16 expression was also examined in MS brain and EAE spinal cord lesions. Increased expression of anti-SPAG16 autoantibodies correlate with increased disease severity and worse prognosis.

Thus, in a first embodiment, the disclosure provides a composition comprising at least two different polypeptides comprising a sequence represented by any of SEQ ID NOS:1-8 or a fragment comprising at least five consecutive amino acids derived from SEQ ID NOS:1-8. Such a composition is herein also designated as a biomarker or as a biomarker panel. The SEQ ID NOS:1-8 correspond with the translated amino acid sequences of the antigens retrieved by the selection of phage displayed MS cDNA expression library on MS patient cerebrospinal fluid (CSF). Thus, the translation of the insert of UH-CSFP1.1 corresponds with SEQ ID NO:1, . . . , and the translation of the insert of UH-CSFP1.8 corresponds with SEQ ID NO:8 (see Table 3). The nucleotide sequences that encode SEQ ID NOS:1-8 are depicted in SEQ ID NOS:9-16 (wherein SEQ ID NO:9 encodes SEQ ID NO:1, . . . , and SEQ ID NO:16 encodes SEQ ID NO:8). Thus, a composition comprises at least two different polypeptides wherein such a polypeptide comprises a sequence as depicted by SEQ ID NOS:1, 2, 3, 4, 5, 6, 7 or 8. This means that a polypeptide present in the composition can also be a protein. As an example SEQ ID NO:8 was cloned as a partial 13.3 kDa protein (protein product as detected using SAS). Since SEQ ID NO:8 (corresponding with UH-CSFP1.8) is a fragment of the SPAG16 protein (which full length is 37 kDa) the composition can also comprise the full length SPAG16 protein. According to particular embodiments, the SPAG16 protein, or a polypeptide fragment thereof, is provided bound to a solid support. The composition of the disclosure can also comprise at least two different polypeptides wherein the polypeptides are fragments comprising at least five consecutive amino acids derived from SEQ ID NOS:1, 2, 3, 4, 5, 6, 7 or 8. It is envisaged that five consecutive amino acids derived from SEQ ID NOS:1-8 are sufficient to be recognized as antigens by the auto-antibodies present in, for example, serum or CSF. Although a polypeptide fragment of, e.g., SPAG16 typically is at least five consecutive amino acids, it is also envisaged that the fragment is at least ten consecutive amino acids, at least fifteen consecutive amino acids, at least twenty consecutive amino acids, at least twenty-five consecutive amino acids, at least fifty consecutive amino acids or even at least one hundred consecutive amino acids. According to particular embodiments, the polypeptide fragment of SPAG16 mimics the epitope bound by the autoantibodies. In such cases, it is possible that, although all amino acids of the fragment are present in the SPAG16 protein, they are not present as a contiguous chain of amino acids, but are presented as the same 3D epitope recognized and bound by the autoantibodies.

In a particular embodiment, the composition comprises eight different polypeptides comprising a sequence selected from SEQ ID NOS: 1-8 or eight different fragments comprising at least five consecutive amino acids derived from SEQ ID NOS:1-8.

In another particular embodiment, the composition comprises four different polypeptides comprising a sequence represented by SEQ ID NOS:4, 5, 6 and 7 or four different fragments comprising at least five consecutive amino acids derived from SEQ ID NOS:4, 5, 6 and 7.

In another embodiment, the disclosure provides the use of a composition of the disclosure for detecting the presence of specific antibodies to at least one polypeptide present in the composition wherein the antibodies are present in a body fluid of a mammal.

In another particular embodiment, the disclosure provides the use of a composition of the disclosure for detecting the presence of specific auto-antibodies to at least one polypeptide present in the composition wherein the auto-antibodies are present in a body fluid of a mammal. In particular embodiments, the use of a composition is an “in vitro” use of a composition. The latter implies a diagnostic method with no direct interaction with the patient.

Also, methods of detecting the presence and/or quantity of specific antibodies to SPAG16 are provided, entailing the steps of contacting a body fluid of a mammal with SPAG16 protein or a polypeptide fragment of at least five consecutive amino acids thereof, and detecting the presence or measuring the quantity of the specific antibodies bound to the SPAG16 protein or polypeptide fragment.

Typically, the detection of at least one specific antibody in a body fluid of a mammal is indicative for multiple sclerosis. Alternatively, the increased quantity of at least one specific antibody in a body fluid of a mammal (e.g., as compared to a non-diseased control) is indicative for multiple sclerosis.

According to further particular embodiments, the SPAG16 fragment corresponds to SEQ ID NO:8. According to still further particular embodiments, the methods additionally comprise a step of detecting the presence or quantity of at least one antibody that has a specificity for a polypeptide comprising a sequence selected from the group represented by SEQ ID NOS:1-7 or for a fragment comprising at least five consecutive amino acids derived from SEQ ID NOS:1-7.

The teem “body fluid” includes blood, blood serum, blood plasma, saliva, urine, tears, bone marrow fluid, cerebrospinal fluid (CSF), synovial fluid, lymphatic fluid, amniotic fluid, nipple aspiration fluid and the like. Preferred body fluids for analysis are those that are conveniently obtained from patients, particularly preferred body fluids include blood serum, blood plasma and CSF.

In yet another embodiment, the disclosure provides a method for detecting multiple sclerosis in a mammal comprising i) detecting the presence of at least one antibody in a body fluid derived from the mammal wherein the antibody has a specificity for a polypeptide comprising a sequence selected from the group consisting of SEQ ID NOS:1-8 or a fragment comprising at least five consecutive amino acids derived from SEQ ID NOS:1-8 and wherein ii) the presence or quantity of the antibody indicates that the mammal suffers from multiple sclerosis.

Likewise, methods of diagnosing the presence of multiple sclerosis in a mammal are provided, comprising contacting a body fluid of a mammal with SPAG16 protein or a polypeptide fragment of at least five consecutive amino acids thereof, and detecting the presence or measuring the quantity of the specific antibodies bound to the SPAG16 protein or polypeptide fragment, wherein the presence of and/or increased quantity of bound antibody as compared to a negative control is indicative of multiple sclerosis.

In yet another embodiment, the method for detecting multiple sclerosis in a mammal of the disclosure is combined with the detection of the MS markers described in US 2004/0043431 and, more specifically, to the markers described in the claims 5, 6, 7, 8, 9 and 10 of the application.

In yet another embodiment, the disclosure provides a method for evaluating the prognosis/disease severity of multiple sclerosis in a mammal comprising i) detecting the presence or quantity of at least one antibody in a body fluid derived from the mammal wherein the antibody has a specificity for a polypeptide comprising a sequence selected from the group consisting of SEQ ID NOS:1-8 or a fragment comprising at least five consecutive amino acids derived from SEQ ID NOS:1-8 and wherein ii) the increased or decreased concentration of the antibody indicates the prognosis of multiple sclerosis in the mammal.

According to specific embodiments, methods for evaluating the prognosis and/or disease severity of multiple sclerosis in a subject are provided comprising contacting a body fluid of a mammal with SPAG16 protein or a polypeptide fragment of at least five consecutive amino acids thereof, and detecting the presence or measuring the quantity of the specific antibodies bound to the SPAG16 protein or polypeptide fragment, wherein the decreased or increased concentration of at least one SPAG16 antibody indicates the prognosis of multiple sclerosis in the patient. In such cases, increased levels of SPAG16 antibody correlates with a worse prognosis (increased severity), while absence or decreased levels of SPAG 16 correlates with a better prognosis (less severe disease). According to specific embodiments, disease prognosis can be measured over time, i.e., the levels of SPAG16 are monitored repeatedly on separate instances to see whether they increase or decrease over time. In such cases, the previous or initial levels will typically serve as a reference or control.

In yet another embodiment, the disclosure provides a method for selecting mammals for a specific therapeutic treatment of multiple sclerosis or evaluating the therapeutic treatment of multiple sclerosis in a mammal comprising i) detecting the presence or quantity of at least one antibody in a body fluid derived from the mammal wherein the antibody has a specificity for a polypeptide comprising a sequence selected from the group consisting of SEQ ID NOS:1-8 or a fragment comprising at least five consecutive amino acids derived from SEQ ID NOS:1-8 and wherein ii) the increased or decreased concentration of the antibody leads to an election of a specific therapeutic treatment of multiple sclerosis in the mammal.

Also, methods are provided for selecting a patient for a specific therapeutic treatment of multiple sclerosis or evaluating the therapeutic treatment of multiple sclerosis in a patient comprising contacting a body fluid of a mammal with SPAG16 protein or a polypeptide fragment of at least five consecutive amino acids thereof, and detecting the presence or measuring the quantity of the specific antibodies bound to the SPAG16 protein or polypeptide fragment, wherein the presence or quantity of the antibody leads to an election of a specific therapeutic treatment of multiple sclerosis in the patient. Typically, such treatments will be treatments used in rapidly evolving MS (e.g., second line treatments, disease-modifying drugs, or treatments for relapsing remitting MS). Examples include, but are not limited to, fingolimod (GILENYA®), natalizumab (TYSABRI®), Mitoxantrone hydrochloride (NOVANTRONE®), BG-12 (TECFIDERA®), Teriflunomide (AUBAGIO®), and Alemtuzumab (LEMTRADA®). Combinations thereof, or combinations of these drugs with first-line medication like beta-interferon treatment or glatiramer are also envisaged.

In a preferred embodiment, the body fluid is CSF. In yet another preferred embodiment, the body fluid is serum. In another preferred embodiment, the mammal is a human.

In yet another embodiment, the disclosure provides an antibody that specifically binds to a polypeptide selected from the group comprising of polypeptides selected from the group consisting of SEQ ID NOS:1-8 or a fragment comprising at least five consecutive amino acids derived from SEQ ID NOS:1-8. Methods for generating antibodies are well known in the art. In a preferred embodiment, the antibodies are monoclonal antibodies. For the purpose of generation of antibodies, the polypeptides forming part of the compositions of the disclosure may be synthesized chemically or may be made in a recombinant way. They may also be coupled to a soluble carrier after synthesis or after recombinant production. If a carrier is used, the nature of such a carrier should be such that it has a molecular weight greater than 5000 and should not be recognized by antibodies. Such a carrier can be a protein. Proteins that are frequently used as carriers are keyhole limpet hemocyanin, bovine gamma globulin, bovine serum albumin, and poly-L-lysine. There are many well described techniques for coupling peptides to carriers. The linkage may occur at the N-terminus, C-terminus or at an internal site in the peptide. The polypeptide may also be derivatized for coupling. The polypeptides may also be synthesized directly on an oligo-lysine core in which both the alpha as well as the epsilon-amino groups of lysines are used as growth points for the polypeptides. The number of lysines comprising the core is preferably three or seven. Additionally, a cysteine may be included near or at the C-terminus of the complex to facilitate the formation of homo- or heterodimers.

According to a further aspect, diagnostic kits are provided for the detection of multiple sclerosis. These kits typically comprise SPAG16 protein or a polypeptide fragment of at least five consecutive amino acids thereof, reagents for making a medium appropriate for an immunological reaction to occur and reagents enabling to detect the antigen/antibody complex that has been produced by the immunological reaction. The kits may further contain an antibody specific for SPAG16. Ways of generating anti-SPAG16 antibodies are known in the art, but also described in the Examples section.

In general terms, the disclosure relates to a process for detecting antibodies related to MS in a biological sample of a mammal liable to contain them, this process comprising contacting the biological sample with a composition according to the disclosure under conditions enabling an immunological reaction between the composition and the antibodies that are possibly present in the biological sample and the detection of the antigen/antibody complex that may be formed. The detection can be carried out according to any classical process. By way of examples, immunoenzymatic processes according to the ELISA technique or immunofluorescent or radioimmunological (RIA) or the equivalent ones (e.g., LINE blot or LINE assay) can be used. Thus, the disclosure also relates to polypeptides according to the disclosure labeled by an appropriate label of the enzymatic, fluorescent, biotin, radioactive type. Such a method for detecting antibodies related to MS comprises, for instance, the following steps: deposit of determined amounts of a polypeptidic composition according to the disclosure on a support (e.g., into wells of a titration microplate), introduction on the support (e.g., into wells) of increasing dilutions of the body fluid (e.g., CSF) to be diagnosed, incubation of the support (e.g., microplate), repeated rinsing of the support (e.g., microplate), introduction on the support labeled antibodies that are specific for immunoglobulins present in the body fluid, the labeling of these antibodies being based on the activity of an enzyme that is selected from among the ones that are able to hydrolyze a substrate by modifying the absorption of the radiation of this latter at least at a given wave length, detection by comparing with a control standard of the amount of hydrolyzed substrate.

In yet another embodiment, the disclosure also relates to a process for detecting and identifying antigens of MS in a body fluid liable to contain them, this process comprising: contacting the biological sample with an appropriate antibody of the disclosure (i.e., antibodies with a specificity for a polypeptide of the composition) under conditions enabling an immunological reaction between the antibody and the antigens of MS that are possibly present in the biological sample and the detection of the antigen/antibody complex that may be formed.

Thus, antibodies, in particular, auto-antibodies, that recognize the polypeptides of the disclosure, can be detected in a variety of ways. One method of detection is further described in the examples and uses enzyme-linked immunosorbant assay (ELISA) of the polypeptides of the disclosure displayed by phages (i.e., phage-ELISA technology). The latter technology is fully described in V. Somers et al. (2005), J. of Autoimmunity 25:223-228, wherein paragraph 2.6 on page 225 is herein specifically incorporated). In other ways in the detection in ELISA a polypeptide or a mixture of polypeptides is bound to a solid support. For example, SPAG16 or a polypeptide fragment thereof may be provided bound to a solid support. In some cases, this will be a microtiter plate but may in principle be any sort of insoluble solid phase (e.g., glass, nitrocellulose). In one embodiment, a suitable dilution or dilutions of, for example, CSF or serum to be tested is brought into contact with the solid phase to which the polypeptide is bound. In another embodiment “a solution hybridization” is carried out in which high affinity interactions occur (e.g., biotinylated polypeptides of the composition are pre-incubated with CSF). The incubation is carried out for a time necessary to allow the binding reaction to occur. Subsequently, unbound components are removed by washing the solid phase. The detection of immune complexes (i.e., auto-antibodies present in, for example, human CSF binding to at least one polypeptide of the disclosure) is achieved using antibodies that specifically bind to human immunoglobulins, and that have been labeled with an enzyme, preferably but not limited to either horseradish peroxidase, alkaline phosphatase, or beta-galactosidase, which is capable of converting a colorless or nearly colorless substrate or co-substrate into a highly colored product or a product capable of forming a colored complex with a chromogen. Alternatively, the detection system may employ an enzyme that, in the presence of the proper substrate(s), emits light. The amount of product formed is detected either visually, spectrophotometrically, electrochemically, fluorescently or luminometrically, and is compared to a similarly treated control. The detection system may also employ radioactively labeled antibodies, in which case, the amount of immune complex is quantified by scintillation counting or gamma counting. Other detection systems that may be used include those based on the use of protein A derived from Staphylococcus aureus Cowan strain I, protein G from group C Staphylococcus sp. (strain 26RP66), or systems that make use of the high affinity biotin-avidin or streptavidin binding reaction.

The polypeptides of the disclosure may be either labeled or unlabeled. Labels that may be employed may be of any type, such as enzymatic, chemical, fluorescent, luminescent, or radioactive. In addition, the polypeptides may be modified for binding to surfaces or solid phases, such as, for example, microtiter plates, nylon membranes, glass or plastic beads, and chromatographic supports such as cellulose, silica, or agarose. The methods by which polypeptides can be attached or bound to solid support or surface are well known to those skilled in the art.

The polypeptides of the disclosure can be prepared according to the classical techniques in the field of peptide synthesis. The synthesis can be carried out in homogeneous solution or in solid phase. For instance, the synthesis technique in homogeneous solution that can be used is the one described by Houbenweyl in the book titled “Methode der organischen chemie” (Method of Organic Chemistry) edited by E. Wunsh, vol. 15-I et II. THIEME, Stuttgart 1974. The polypeptides of the disclosure can also be prepared in solid phase according to the method described by Atherton & Shepard in their book titled “Solid phase peptide synthesis” (Ed. IRL Press, Oxford, N.Y., Tokyo, 1989). Synthesis protocols in the art generally employ the use of t-butyloxycarbonyl- or 9-fluorenylmethoxy-carbonyl-protected activated amino acids. The procedures for carrying out the syntheses, the types of side-chain protection, and the cleavage methods are amply described in, for example, Stewart and Young, Solid Phase Peptide Synthesis, 2nd Edition, Pierce Chemical Company, 1984; and Atherton and Sheppard, Solid Phase Peptide Synthesis, IRL Press, 1989.

In yet another embodiment, antibodies raised to polypeptides of the disclosure (or carrier-bound polypeptides) can also be used in conjunction with labeled polypeptides of the disclosure for the detection of (auto)-antibodies present in serum or CSF by competition assay. In this case, antibodies raised to polypeptides are attached to a solid support that may be, for example, a plastic bead or a plastic tube. Labeled polypeptide is then mixed with suitable dilutions of the fluid (e.g., CSF) to be tested and this mixture is subsequently brought into contact with the antibody bound to the solid support. After a suitable incubation period, the solid support is washed and the amount of labeled polypeptide is quantified. A reduction in the amount of label bound to the solid support is indicative of the presence of (auto)-antibodies in the original sample. By the same token, the polypeptide may also be bound to the solid support. Labeled antibody may then be allowed to compete with (auto)-antibody present in the sample (e.g., CSF) under conditions in which the amount of polypeptide is limiting. As in the previous example, a reduction in the measured signal is indicative of the presence of (auto)-antibodies in the sample tested.

In a particular embodiment, a test for giving evidence of the fact that one or more polypeptides present in a composition of the disclosure are recognized by antibodies present in, for example, CSF of serum (for example auto-antibodies present in CSF of multiple sclerosis patients) is an immunoblotting (or Western blotting) analysis. In the latter case polypeptides can be chemically synthesized or polypeptides (or the protein) can be produced via recombinant techniques. In short, after sodium dodecyl sulfate-polyacrylamide gel electrophoresis, polypeptides of the disclosure are blotted onto nitrocellulose membranes (e.g., Hybond C. (Amersham)) as described by H. Towbin et al., 1979, “Electrophoretic transfer of proteins from polyacrylamide gels to nitrocellulose sheets: procedure and some applications,” Proc. Nall. Acad. Sci. USA 76:4350-4354. In order to identify selective recognition of polypeptides (or proteins) of the disclosure by CSF, nitrocellulose sheets are incubated overnight with each of these samples (e.g., diluted 1:50) (after blocking a-specific protein-binding sites). Reactive areas on the nitrocellulose sheets are revealed by incubation with, e.g., peroxidase conjugated goat anti-human immunoglobulin G antibody (e.g., diluted 1:200) for four hours, and after repeated washings, color reaction is developed by adding, for example, alpha-chloronaphtol (Bio-Rad Laboratories, Richmond, Calif.) in the presence of hydrogen peroxide.

It goes without saying that the free reactive functions that are present in some of the amino acids, which are part of the constitution of the polypeptides of the disclosure, particularly the free carboxyl groups that are carried by the groups Glu and Asp or by the C-terminal amino acid on the one hand and/or the free NH₂ groups carried by the N-terminal amino acid or by amino acids inside the peptidic chain, for instance, Lys, on the other hand, can be modified in so far as this modification does not alter the above mentioned properties of the polypeptide. The polypeptides that are thus modified are naturally part of the disclosure. The above mentioned carboxyl groups can be acylated or esterified. Other modifications are also part of the disclosure. Particularly, the amine or carboxyl functions or both of terminal amino acids can be themselves involved in the bond with other amino acids. For instance, the N-terminal amino acid can be linked to the C-terminal amino acid of another peptide comprising from one to several amino acids.

Furthermore, any peptidic sequences resulting from the modification by substitution and/or by addition and/or by deletion of one or several amino acids of the polypeptides according to the disclosure are part of the disclosure insofar as this modification does not alter the above mentioned properties of the polypeptides. The polypeptides according to the disclosure can be glycosylated or not, particularly in some of their glycosylation sites of the type Asn-X-Ser or Asn-X-Thr, X representing any amino acid.

An advantageous recombinant polypeptide included in the composition of the disclosure is SEQ ID NO:6 since this polypeptide shows the highest frequency of antibody responses in CSF of MS patients with no reactivity in the control patients.

Variations of these polypeptides are also possible depending on its intended use. For example, if the polypeptide is to be used to raise antisera, the polypeptide may be synthesized with an extra cysteine residue added. This extra cysteine residue is preferably added to the amino terminus and facilitates the coupling of the polypeptide to a carrier protein, which is necessary to render the small polypeptide immunogenic. If the polypeptide is to be labeled for use in radioimmune assays, it may be advantageous to synthesize the protein with a tyrosine attached to either the amino or carboxyl terminus to facilitate iodination. This polypeptide possesses, therefore, the primary sequence of the polypeptide above-mentioned but with additional amino acids that do not appear in the primary sequence of the protein and whose sole function is to confer the desired chemical properties to the polypeptide.

In yet another embodiment, the disclosure provides for a kit to diagnose MS. To carry out the diagnostic method for MS, the following necessary or kit can be used, the necessary or kit comprising: a composition (comprising at least one polypeptide selected from SEQ ID NOS:1-8) according to the disclosure, or at least one fragment comprising at least five consecutive amino acids derived from SEQ ID NOS:1-8, reagents for making a medium appropriate for the immunological reaction to occur, reagents enabling to detect the antigen/antibody complex that has been produced by the immunological reaction, the reagents possibly having a label, or being liable to be recognized by a labeled reagent, more particularly in the case where the above mentioned polypeptide is not labeled.

Other characteristics and advantages of the disclosure will appear in the following examples and the figures illustrating the disclosure.

### EXAMPLES

### Example 1

### Enrichment of a Phage Displayed MS cDNA Library with MS CSF

To create an MS cDNA display library, a normalized cDNA library derived from active, chronic MS plaques, with varying degrees of demyelination and inflammatory activity, which was originally cloned into the pT7T3-Pac vector, was cloned into the M13 filamentous phage display vectors pSPA, B and C. These vectors allow expression of cDNA products (peptides) derived from MS brain plaques as a fusion to minor coat protein pVI of filamentous phage M13 in three reading frames for correct expression of the protein products. A total library size of 1.1×10⁷ colony forming units (cfu) was obtained.

To enrich the MS cDNA display library for cDNA products (displayed peptides) that are specifically bound by autoantibodies present in cerebrospinal fluid of MS patients, we performed successive rounds of selection (see FIG. 3 and Table 4) on pooled CSF of ten randomly selected RR MS patients. Following rescue of the phage clones after each of four rounds of selection, enriched phage clones, each bearing a single fusion peptide derived from the MS cDNA display library, were randomly selected for further study.

### Example 2

### Characterization of the Enriched Phage Clones

Among the enriched clones, a total of 52 clones were selected. cDNA inserts were sequenced and the translated protein sequences were determined. Sequence analysis revealed eight antigenic targets, which we annotated with the name UH-CSFP-number, which is short for University Hasselt—cerebrospinal fluid pool—number of the clone. These sequences corresponded to known proteins expressed in the correct reading frame, but also homology to untranslated regions of expressed genes, such as 3′ UTR sequence of proteolipid protein, or homology to out of frame sequences were obtained (see Table 3).

In initial experiments, we assessed the reactivity of the individual MS CSF specimens used for the selection procedure against eight enriched antigenic cDNAs. As shown in Table 4, of the ten CSF samples from RR-MS patients, eight contained antibodies that reacted with at least two phage-peptide clones. These clones were used for subsequent screening on a large panel of CSF from other MS patients as well as CSF from patients with other inflammatory (OIND) and non-inflammatory neurological disorders (NIND).

Of the ten RR-MS patients used for the selection procedure, paired serum samples were also collected and used for screening for antibody reactivity towards the eight enriched antigenic cDNAs. In three out of eight patients with antigen-specific antibodies present in CSF, reactivity toward one of the eight antigenic cDNAs was also found in paired serum. There was a good association between the positive signal observed in the CSF, and the reactivity demonstrated in serum. The signal on individual CSF tested was higher than that on individual serum tested, which is consistent with the dilution of the antigen specific antibodies present in the serum, when antibodies are intrathecally produced. When low reactivity in the CSF was observed, no positive signal was found in the serum of the same patient. In addition, no reactivity in paired serum samples was demonstrated in patients with antibody negative CSF (data not shown).

### Example 3

### Detailed Serological Analysis of the MS Panel

Next, clones were tested on a large panel of individual CSF specimens not used for the selection procedure (n=63 for MS patients (54 RR-MS, three SP-MS patients and six PP-MS), n=30 for OIND patients and n=64 for NIND patients). The results of the phage ELISA screening of the individual phage-cDNA clones on 167 different CSF are presented in Table 5. All antigens tested showed exclusive or preferential reactivity in the MS group as compared to the control group. Clones UH-CSFP1.4-UH-CSFP1.7 showed reactivity in 17 of 73 (23%) MS CSF whereas no reactivity towards the OIND and NIND CSF specimens was observed. The remaining clones (UH-CSFP1.1-UH-CSFP1.3 and UH-CSFP1.8) showed higher reactivity in the MS group 25/73 (34%) as compared to the control group 13/94 (14%), and therefore, these clones were also defined as clones with an MS-related serological profile.

In total, 33 of 73 (45%) MS patients showed CSF IgG antibodies reactive with at least one of the panel of eight antigenic targets. The highest frequency of antibody responses in MS CSF with no reactivity in the control group was found to UH-CSFP1.6. All CSF samples tested showed equivalent total CSF IgG levels. CSF samples with high IgG concentration were normalized to the normal CSF concentration range.

### Example 4

### Expression Pattern of Novel MS Markers

Northern blot analysis of the antigenic targets with no reactivity in the control group was performed on a variety of normal human tissues. UH-CSFP 1.4 gave a transcript of 1.9 kb and was highly expressed brain, heart and placenta, and to a lower extent in skeletal muscle, kidney and liver. UH-CSFP1.7 gave a transcript size of 5.1 kb and showed a high expression in brain, heart and skeletal muscle. No transcript could be detected for UH-CSFP1.5 and UH-CSFP1.6.

We further selected four of the antigenic targets (UH-CSFP1.3, UH-CSFP1.6, UH-CSFP1.7 and UH-CSFP1.8), for protein expression in E. coli. UH-CSFP1.7 was cloned as antigen (6.1 kDa) and the UH-CSFP1.8 (SPAG16) protein was cloned as partial (13.3 kDa, protein product as detected using SAS) and full-length protein (20.3 kDa) with a 16.7 kDa thioredoxin fusion (His tag) resulting in 22.8 kDa protein for UH-CSFP1.7 and 30 kDa partial and 37 kDa full length band for SPAG16 protein on SDS-PAGE after Coommassie staining (see FIG. 2).

Due to the presence of amber stop codons in the sequences of clones UH-CSFP1.3 and UH-CSFP1.6, site-directed mutagenesis was performed to create glutamine codon(s) for use in bacterial protein expression in the non-suppressing LMG194 strain. Following site-directed mutagenesis, UH-CSFP1.3 was cloned as antigen (6.11 kDa) resulting in a 22.8 kDa protein including thioredoxin (see FIG. 6). Due to toxicity, the entire UH-CSFP1.6 could not be expressed. Therefore, the first part of the protein encoded by amino acids 1-52 of the antigen (as detected using SAS) was produced, resulting in a 22.5 kDa protein product including thioredoxin (see FIG. 6).

### Example 5

### Autoantibody Reactivity and Clinical Data

We next determined whether reactivity to our antigenic panel was associated with a particular disease phenotype. Autoantibody reactivity to at least one of the eight antigenic targets was demonstrated in 30/64 (47%) RR-MS patients, 3/6 (50%) PP-MS patients and 0/3 SP-MS patients. Demographic variables and EDSS score in antibody-positive and antibody-negative MS patients are shown in Table 6. No differences were observed in age between antibody-positive and antibody-negative patients. Antibody reactivity could be observed in some patients at time of diagnosis and was present in patients with short disease duration (<1 year), but also in patients with a disease duration greater than ten years. However, no correlation was found between antibody reactivity and disease duration.

In order to assess the influence of antibody reactivity on disease severity, we examined the relationship between antibody reactivity and EDSS score. Antibody reactivity was found in 21/50 (42%) of patients with EDSS<3, 6/11 (54%) of patients with EDSS=3 or 3.5, and 3/5 (60%) of MS patients with EDSS=4. Although a higher percentage of patients showed reactivity to the panel of eight antigenic cDNAs with increasing EDSS score, this difference was not significant.

### Example 6

### Solution Phase Assay/Competition ELISA

To determine whether the observed autoantibody signature of MS CSF is due to the MS brain plaque derived peptides, two MS CSF specimens (one positive (MS-CSF8) and one negative (MS-CSF26) for UH-CSFP 1.1 were pre-incubated with the synthetic peptide UH-CSFP1.1 (NH₂-ASSRGYEDLRTF-COOH) representing the cDNA insert of clone UH-CSFP1.1 and with a non-specific (random) peptide. As shown in FIG. 4, preincubation with UH-CSFP1.1 peptide clearly inhibited the formation of specific IgG antibody/phage UH-CSFP1.1 complexes for MS-CSF8 while no inhibition was found for MS-CSF26. In contrast, CSF reactivity against clone UH-CSFP 1.1 was not inhibited by addition of the random peptide.

### Example 7

### Monoclonal Antibody Production

A murine monoclonal antibody for UH-CSFP1.1 was produced based on the hybridoma technology developed by Kohler and Milstein.⁵⁵ FIG. 5 represents antibody reactivity against UH-CSFP1.1 peptide following hybridoma selection. The ODs of supernatants from ten random tested clones of hybridomas are indicated at first screening for antibody production. A positive ELISA signal was obtained for clone 7. Further subcloning of this clone resulted in a monoclonal hybridoma cell line producing antibodies directed against UH-CSFP1.1 peptide. The produced monoclonal antibody showed the same epitope specificity as previously identified for MS serum or CSF samples. This allows further analysis of the UH-CSFP1.1 antigen. In an alternative approach, we are using phage particles expressing the UH-CSFP antigenic targets for immunization of Balb/c female mice. Advantages of using phage-displayed peptides is that they are cheap, easy to obtain and that the antigen is displayed to the murine immune system as it is recognized in serum or CSF from MS patients.

### Example 8

### ELISA on Peptides

To address whether antibody reactivity was also observed against linear peptides, we used ELISA on synthetic peptides (UH-CSFP1.1, UH-CSFP1.2, UH-CSFP1.4 and UH-CSFP1.5). As shown in FIG. 7, MS patient No. 4 showed CSF reactivity against clone UH-CSFP1.1, while for the other peptides, no reactivity was found. For the other MS patients, no reactivity was seen against any of the tested peptides. These results were consistent with the phage ELISA results for UH-CSFP1.1, UH-CSFP1.2, UH-CSFP1.4 and UH-CSFP1.5.

### Example 9

### ELISA on Purified Recombinant Proteins

After protein expression of UH-CSFP1.3, UH-CSFP1.6, UH-CSFP1.7 and UH-CSFP1.8 (as described in Example 4, second paragraph), immunoreactivity for each purified recombinant protein was measured in serum. FIG. 8 represents antibody reactivity towards UH-CSFP1.6 in serum from 16 randomly selected MS patients, 15 NIND/OIND patients and 16 healthy controls. Reactivity was demonstrated in 3/16 MS patients and 1/15 NIND/OIND patients, while no reactivity was found in healthy controls.

### Example 10

### Immunohistochemical Staining

After monoclonal antibody production (Example 7), the murine monoclonal antibody against UH-CSFP1.1 was used for immunohistochemical staining of experimental autoimmune encephalomyelitis (EAE) rat brain tissue. It was observed that the murine monoclonal antibody against UH-CSFP 1.1 stained the endothelial lining of blood vessels and showed cytoplasmic staining of large neurons.

### Example 11

### SPAG16 is an Autoantibody Target in CSF and Plasma of MS Patients

One of the phage clones, UH-CSFP1.8, represents the Homo sapiens sperm-associated Ag 16 (SPAG16) protein, which is a novel antigen in MS. The anti-SPAG16 antibodies identified in the CSF of a subgroup of MS patients are directed against an epitope present in the last 121 aa of SPAG16-2. Using BLAST analysis, this epitope was also found to be present in SPAG16-1 (FIG. 9, Panel A), and other putative isofouns. Indeed, monoclonal antibodies directed against SPAG16-2 also bind SPAG16-1, as indicated by western blot of a testis lysate (FIG. 9, Panel B).

Isoelectric focusing (IEF) was used to detect SPAG16-specific oligoclonal bands (OCBs) in the CSF of MS patients, indicating a persistent IgG response towards the target. After separation, IgGs were blotted onto membranes coated with recombinant SPAG16 or the control protein thioredoxin (THIO), for which the purity was confirmed (FIG. 9, Panel C). We found that two out of the four MS patients tested, had specific OCBs binding SPAG16 and not THIO (FIG. 10, Panel A), indicating an oligoclonal intrathecal IgG response towards SPAG16 in these MS patients.

We also detected a deviating humoral immune response in the blood of MS patients. A recombinant in-house developed protein ELISA was used for anti-SPAG16 immunoreactivity screening in plasma samples from MS patients (n=153), clinically isolated syndrome patients (CIS; n=101), patients with non- and other inflammatory neurological diseases (NIND; n=51; OIND; n=22) and healthy controls (n=204). Elevated plasma antibodies (above the receiver operating characteristic (ROC) determined cut-off) against SPAG16 were detected in 21% (32/153) of MS patients with a 95% specificity (11/204 healthy controls) for the disease (FIG. 10, Panel B). Elevated antibody levels against SPAG16 were detected in a significantly larger proportion of MS patients compared to all other groups; namely CIS patients (15/101; P<0.01), neurologic controls (NIND; 3/51; P<0.01, OIND; 3/22; P<0.05) and healthy controls (11/204; P<0.001). Anti-SPAG16 antibodies could be detected in the CIS patient group (15/101; 15%; FIG. 9, Panel B), indicating that these antibodies are already present in the early disease stages in a subgroup of patients. There was however no correlation between the presence of anti-SPAG16 antibodies and the conversion from CIS to MS. The relative frequency of anti-SPAG16 antibody positive MS patients varied between relapsing-remitting (RR)-MS (18/70; 26%), secondary-progressive (SP)-MS (12/58; 21%) and primary-progressive (PP)-MS (2/25; 8%). We observed no correlation between anti-SPAG16 antibody positivity and age, gender, clinical characteristics or characteristics of the CSF, although this study was neither designed nor powered to provide a test for correlation. No differences in anti-SPAG16 antibody levels were found for the majority of MS patients during relapse or remission (n=48; data not shown), indicating continuous anti-SPAG16 antibody production. Upon anti-SPAG16 antibody isotyping (n=22), we observed that most patients had IgG1, either without the presence of other isotypes or in combination with IgG3 and to a lesser extent IgG2 or IgG4 (FIG. 10, Panel C). Since IgG1 and IgG3 are activators of the complement cascade, anti-SPAG16 antibodies could activate the complement system, indicating their pathogenic potential.

### Example 12

### Anti-SPAG16 Antibodies Exacerbate MOG-Peptide Induced EAE

Next, to study the pathogenic relevance of circulating anti-SPAG16 antibodies, we investigated the effect of passive antibody transfer of anti-SPAG16 mAbs in the chronic MOG³⁵⁻⁵⁵ peptide-induced EAE model. First, using BLAST analysis we determined whether the 121 aa initially identified using SAS are present in murine SPAG16 isoforms (FIG. 11, Panel A). These 121 aa were present in murine SPAG16 isoform 1 and 3, but not in isoform 2. Next, we confirmed that our in-house produced mouse mAbs against human SPAG16 (binding both isoform 1 and 2; FIG. 9, Panel B) also bind murine SPAG16 in the spinal cord. Indeed, a SPAG16 isoform of ˜47 kDa was identified, corresponding to isoform 3 in mice (FIG. 11, Panel B). Furthermore, the mouse mAbs against SPAG16 were shown to be representative for the immunoreactivity seen in MS patients, since there was competition between the mouse mAbs and anti-SPAG16 antibody-positive MS plasma (FIG. 11, Panel C).

Passive transfer of the mAbs was performed at the onset of disease (score 0.5-1) allowing for the transport of the injected mAbs across a compromised blood-brain barrier. As shown in FIG. 12, Panel A, anti-SPAG16 mAbs significantly exacerbated EAE, indicated by a higher mean EAE score (overall disease course: repeated measures ANOVA, P<0.01) compared to the control group. This was also reflected in the decreased weight of the animals treated with anti-SPAG16 mAbs (FIG. 12, Panel B). The mean cumulative score (P<0.01) and mean number of follow-up days with a disease score of at least 2 (P<0.01) were significantly higher in the anti-SPAG16 antibody receiving animals (Table 9).

To explore the mechanism by which anti-SPAG16 mAbs mediated EAE exacerbation, IHC staining of spinal cord tissue from mice that received anti-SPAG16 (FIG. 12, Panel C) or isotype control mAbs (FIG. 12, Panel D) was performed (17 days post-antibody transfer). IHC images were quantified for inflammatory cell infiltration (FIG. 12, Panels E and F), demyelination (FIG. 12, Panel G), astrogliosis (FIG. 12, Panel H) and bound immunoglobulins (FIG. 12, Panels I and J). Taken together, we observed an increased number of macrophages (P<0.05) and enhanced immunoglobulin deposition (P<0.05) within the spinal cords of animals injected with anti-SPAG16 mAbs, indicating that these antibodies bind their target in the mouse spinal cord and mediate enhanced inflammatory cell infiltration. As no obvious effect on myelin basic protein (MBP) loss is seen, the exacerbating effect is most likely not mediated by increased myelin degradation by bound antibodies and associated macrophages. The data clearly show that an increased presence of anti-SPAG16 antibodies correlates with a worse prognosis.

### Example 13

### SPAG16 is Up-Regulated in Astrocytes within MS Brain and EAE Spinal Cord Lesions

To determine whether SPAG16 was expressed in MS brain tissue, we performed immunohistochemistry (IHC) on MS (n=7) and control (n=5) brain tissue with anti-SPAG16 antibodies (FIG. 13). Low levels of SPAG16 were detected in the white matter of control brains (FIG. 13, Panel A), and in neurons in the grey matter (FIG. 13, Panels C and D). In MS brain tissue, identification of lesions was based on IHC for the absence of myelin (FIG. 13, Panel E) and activated microglia/macrophages (FIG. 13, Panel F). In MS lesions, SPAG16 was up-regulated (FIG. 13, Panels G and I) compared to normal appearing white matter tissue of MS patients and normal white and grey matter in controls (FIG. 13, Panels A and C). Most intense staining was demonstrated in astrocytes in the center of active lesions (FIG. 13, Panels I and J). At the lesion edge, SPAG16 expression was still detectable in astrocytes (FIG. 13, Panel G), but less intense. Double-staining for SPAG16 and glial fibrillary acidic protein (GFAP) (FIG. 13, Panel H) confirmed SPAG16 expression in astrocytes. In controls we found a few, weakly SPAG16-positive astrocytes, which is in accordance with the staining of SPAG16 in The Human Protein Atlas, where glial cells do not stain positive.³⁹

To study whether SPAG16 expression is also increased in an animal model for MS, we induced EAE in C57BL/6J mice using myelin oligodendrocyte glycoprotein (MOG) peptide, followed by IHC analysis of both healthy and EAE spinal cords. In healthy mouse spinal cord (n=4), consistent with human control brain tissue, a low level of SPAG16 expression was observed in neurons (FIG. 13, Panel K). In EAE (n=5), spinal cord lesions were identified by staining for infiltrating macrophages (FIG. 13, Panel N). In accordance to what we found in MS lesions, an up-regulation of SPAG16 was observed in astrocytes at the EAE spinal cord lesion site (FIG. 13, Panels L, M, 0, and P), indicating a similar role for the protein in MS and EAE.

### Discussion

SPAG16 was first identified as an autoantibody target in MS patient CSF. It could additionally be shown that SPAG16 is a protein with pathogenic relevance targeted by the humoral autoimmune response both in CSF and plasma of MS patients. Besides a role as a possible diagnostic marker for MS, the pathogenic potential of the anti-SPAG16 antibodies was demonstrated in vivo. Furthermore, SPAG16 expression was up-regulated in reactive astrocytes, both in MS and EAE lesions, suggesting a similar role of the protein and a possible mechanism for breaking of the immune tolerance.

Specific oligoclonal bands (OCBs) against SPAG16 were detected in a subgroup of MS CSF. OCBs are an important hallmark of MS CSF and are produced in the context of sustained antigenic stimulation in the intrathecal compartment.⁵ Therefore, it is intriguing to find SPAG16-specific OCBs in MS CSF, particularly since in spite of intensive research, the target antigen(s) recognized by individual OCBs in MS have remained elusive.⁴¹ Besides testing for the presence of OCBs against SPAG16 in CSF, the diagnostic potential of anti-SPAG16 antibodies could be reinforced by screening for antibodies in plasma. Elevated levels of anti-SPAG16 antibodies were present in the plasma of 21% of MS patients with 95% specificity. However, we observed a lower percentage of anti-SPAG16 positive PP-MS patients (8%) compared to RR-MS (26%) and SP-MS (21%). This can be explained by the fact that the level of inflammation (including the humoral immune response) is less pronounced in PP-MS patients.⁴² Again, increased levels of SPAG16 correlate with increased inflammation and worse prognosis.

Identification of SPAG16 as a target of the B cell responses associated with MS raises two important questions. What mechanisms drive the anti-SPAG16 humoral immune response and how could antibodies against SPAG16 contribute to the pathogenesis of MS?

Antibody reactivity towards other intracellular targets like SPAG16 (e.g., neurofilament, tubulin) is not uncommon in MS.^{43, 44} There are several hypotheses on the generation of an antibody response towards intracellular proteins.⁴⁵ These include a) general dysregulation of the immune system in autoimmune diseases, b) cell death (e.g., neurons that express a low level of SPAG16) leading to exposure of intracellular antigens such as SPAG16 (epitope spreading), c) molecular mimicry. Moreover, we demonstrated an up-regulation of SPAG16 in MS and EAE lesions in reactive astrocytes, indicating a similar role of the protein in MS and EAE. Additionally, such an altered expression pattern could be a mechanism for breaking of the immune tolerance as exemplified in systemic sclerosis and cancer, where an altered expression pattern of other sperm-associated antigens elicited an autoantibody response.38, 46

As for the role of SPAG16, not much is known about the protein in other diseases or other tissues, especially the brain and spinal cord, but it has become clear that the expression of SPAG16 isoforms is not restricted to sperm cells. Recent data reveal its presence in many tissues, including disease-relevant fibroblast-like synoviocytes in rheumatoid arthritis.⁴⁰ Additionally, the presence of multiple, not yet validated, SPAG16 isoforms further increases the complexity. The observation that SPAG16 is up-regulated in astrocytes within or near the MS lesion is in part reminiscent of the alpha B-crystallin data. Alpha B-crystallin is a stress-induced heat shock protein that is increased within or surrounding MS lesions.⁴⁷ Since an active MS lesion is an area of cellular stress and inflammation, we hypothesize that the up-regulation of SPAG16 in astrocytes is due to local mediators such as pro-inflammatory factors. In these cells, SPAG16 could play a stress-related, a structural or a motility-associated role, since SPAG16-1 is a known motility protein in sperm cells and motile cilia.

Next we were interested in how anti-SPAG16 antibodies could contribute to the pathogenesis of MS. We determined the isotype of the anti-SPAG16 antibodies in 22 MS patients and mostly found IgG1, which is known to activate the complement system. Moreover, the pathogenic potential of anti-SPAG16 antibodies was clearly demonstrated by their exacerbating effect on EAE disease in mice. This exacerbating effect was not due to changes in demyelination but due to an increase in macrophage infiltration and immunoglobulin deposition. Over the past years, many autoantibody targets have been identified in MS such as MOG, neurofascin and recently KIR 4.1, but, in contrast to SPAG16, these targets rarely showed specific antibody reactivity in MS combined with pathogenic potential in vivo:⁴⁸⁻⁵¹

In conclusion, the present study provides the basis for the involvement of SPAG16 and anti-SPAG16 antibodies in a subpopulation of MS patients.

### Materials and Methods

### Patients and Controls

Cerebrospinal fluid samples were obtained from 73 MS patients, 30 patients with other inflammatory (meningitis, polyneuropathy) and 64 patients with non-inflammatory neurological disorders (hernia, epilepsy, dementia, headache, Alzheimer patients, . . . ) undergoing lumbar puncture for diagnostic purposes. MS patients were diagnosed according to the McDonald and Poser criteria.¹⁴ Characteristics of the study population are shown in Table 1. From 28 out of 73 MS patients, paired serum samples were collected. CSF and serum samples were stored at −80° C. after collection. The study was approved by the institutional ethics committee.

An extended cohort was used for the study and validation of SPAG16 as an autoantibody target (Examples 11-13). All samples were collected after approval by the Medical Ethical Committee of Hasselt University (Belgium) and informed consent from study participants was obtained. MS patients were diagnosed according to the McDonald criteria.⁵² In general, CIS patients were clinically followed for two years. Details of these MS patients and control groups are provided in Table 7.

Cloning of an MS cDNA Library for pVI Display and Serological Antigen Selection (SAS) of Phage pVI Displayed cDNA Repertoires

A normalized cDNA library (1.0×10⁶ primary recombinants) derived from three active chronic MS plaques, with varying degrees of demyelination and inflammatory activity (gift from Dr. Soares) was used to construct an MS cDNA display library by cloning it as a fusion protein with filamentous phage minor protein pVI. Therefore, the library was transferred to our phage display vectors, named pSPVIA, pSPVIB and pSPVIC, each encoding one of three reading frames. Details of the cloning procedure are described in reference 15.

The SAS procedure was performed as described previously (J. I. Somers¹⁵). In brief, CSF samples of ten randomly selected untreated relapsing remitting (RR)-MS patients were pooled and used for affinity selections. Characteristics of the patients used for affinity selections are shown in Table 2. Before the start of the selection procedure, CSF samples were absorbed against Escherichia coli (E. coli) and phage antibodies as described in reference 15. Following adsorption, pooled CSF was stored at −20° C. Subsequently, pooled preabsorbed CSF was used for the selection procedure. Affinity selections were performed as described before.¹⁵ In brief, an immunotube (Nunc, Roskilde, Denmark) was coated with rabbit anti-human IgG (Dako, Glostrup, Denmark) in coating buffer (0.1 M sodium hydrogen carbonate pH 9.6) for two hours at 37° C. After washing the immunotube twice with phosphate-buffered saline containing 0.1% TWEEN® 20 and twice with PBS, the tubes were blocked for two hours with 2% MPBS (2% milk powder in PBS). For the first round of the selection procedure, phage were prepared from the MS cDNA library cloned in the three phage display vectors pSP6A, B and C. Phage were prepared as described previously.¹⁶ Approximately 10¹³ phage were added to pooled preabsorbed CSF (1:5 diluted in 4% MPBS) and incubated for 1.5 hour at RT on a rotating platform. After washing the coated immunotube twice with PBST and twice with PBS, the preincubated CSF and phage mix was transferred to the coated immunotube and incubated for 30 minutes on a rotating platform and 120 minutes standing at RT. Tubes were then washed extensively with PBST and PBS to remove non-binding phage. Binding phage were eluted with 100 mM triethylamine and neutralized with 1 M Tris HCl as described before.¹⁷ .E. coli TG1 cells were infected with input and output phage and plated on 2×TY agar plates containing ampicillin and glucose (16 g/l bacto-tryptone, 10 g/l yeast extract, 5 g/l NaCl, 15 g bacto-agar/l, ampicillin at 100 μg/ml and glucose at 2%) at each round of selection. Resultant colonies were scraped and phages were rescued for further rounds of affinity selections. To monitor enrichment of specific clones, input and output phage from each round of selection were titrated and the ratio of output/input phage was determined. After several rounds of selection, individual colonies were selected and the insert size and sequence was determined as described in reference 15. Sequences were submitted to GenBank for BLAST homology search.

### Phage ELISA

ELISA of ligand displaying phage was performed as described in reference 15. Immunoreactivity for each phage peptide was measured in relation to an internal control signal detected by antibody reactivity against the empty phage. For competition ELISA, CSF was pre-incubated in the presence of 0-50 pmol/50 μl synthetic peptide UH-CSFP1.1 (NH₂-ASSRGYEDLRTF-COOH) (SEQ ID NO:1) or random peptide. Subsequently, the immunoreactivity to phage UH-CSFP1.1 was determined according to the standard phage ELISA procedure.

### Northern Blot Analysis

Plasmid was isolated using the Qiagen Plasmid Midi Kit according to the manufacturer's instructions. The isolated plasmid was EcoRI/NotI digested and the excised DNA was gel-purified (GFX™ PCR DNA and Gel Band Purification Kit, GE Healthcare, Brussels, Belgium).

The excised DNA fragment was used as probe in Northern blot. Probes were labelled with [α³²P] using the High Prime DNA Labeling Kit (Roche, Vilvoorde, Belgium). Briefly, 50 ng excised DNA was first denatured during ten minutes in boiling water and immediately chilled on ice. The labelling mix was added to the DNA and after 45 minutes incubation at 37° C., the reaction was stopped by addition of 0.2 M EDTA. Labelled DNA was purified with Sephadex G75 columns and radioactivity measured with a scintillation counter.

Northern blotting was performed using the Multiple Tissue Northern (MTN™) Blot (BD Biosciences, Erembodegem, Belgium). Briefly, labelled DNA or human β-actin cDNA control probe was denatured at 97° C. during five minutes and immediately chilled on ice for a few minutes. After prehybridization of the blotting membrane with ExpressHyb solution, the radioactively labelled probe was added (2-10 ng/ml or 1−2×10⁶ cpm/ml) and hybridization occurred overnight at 68° C. After washing three times, the blotting membrane was exposed to X-ray film at −70° C. and developed using the Gevamatic 60 (Agfa Gevaert, Mortsel, Belgium).

Cloning of Antigenic cDNAs in pBAD/Thio-TOPO Vector and Expression of Recombinant Proteins

Several of the antigenic cDNAs were cloned into the pBAD/Thio-TOPO vector (Invitrogen Life Technologies, Merelbeke, Belgium) and transformed into LMG194 cells according to the manufacturer's directions. Clones were cultured in LB Broth Base medium (Invitrogen Life Technologies, Merelbeke, Belgium) supplemented with ampicillin. Expression in E. coli, driven by the araBAD promoter (p_{BAD}), was induced by addition of 0.2% arabinose. Recombinant proteins were expressed as fusions to His-Patch thioredoxin and were purified by Ni-NTA beads (Qiagen, Venlo, The Netherlands) according to the manufacturer's instructions. Expression of the proteins of the correct size was confirmed by SDS-PAGE. Protein identity was confirmed by mass spectrometry.

Due to the presence of amber stop codons in the nucleotide sequences of clones UH-CSFP1.3 and UH-CSFP1.6, site-directed mutagenesis (Quikchange Site-Directed Mutagenesis Kit, Stratagene) was performed according to the manufacturer's directions in order to create glutamine codon(s) for use in bacterial protein expression in the non-suppressing LMG194 strain. For UH-CSFP 1.6 the first part of the protein encoded by amino acids 1-52 of the antigen (as detected using SAS) was produced.

### Monoclonal Antibody Production

A murine monoclonal antibody for UH-CSFP 1.1 was produced according to the hybridoma technology developed by Kohler and Milstein.⁵⁵ Due to its small size, UH-CSFP1.1 peptide was coupled to keyhole limpit hemocyanin (KLH) as carrier (UH-CSFP1.1) (Eurogentec) for immunization of Balb/c female mice. After three intraperitoneal immunizations with 150 μg UH-CSFP1.1-KLH, spleen cells were isolated and fused with a mouse myeloma cell line (Sp2/0). After selection of fused hybridomas by culturing in HAT medium, screening of the resulting hybridoma cell lines was performed by peptide ELISA using coated UH-CSFP1.1 peptide (Eurogentec) and cell line supernatant. After sub-cloning, a monoclonal hybridoma cell line was obtained, which produced antibodies directed against UH-CSFP1.1 peptide.

### ELISA on Peptides

For ELISA experiments, 96-well ELISA plates (Greiner) are coated with 100 μl of 1 μg/ml peptide (UH-CSFP1.1, UH-CSFP1.2, UH-CSFP1.4 and UH-CSFP1.5) in PBS and kept overnight at RT. Wells are then washed with three times with PBS 0.05% TWEEN®20 and blocked at RT with blocking buffer (2% nonfat milk in PBS). After washing three times with PBS 0.05% TWEEN®20, the plates are incubated with 100 μl diluted samples (CSF 1:5 diluted and serum, 1:100 diluted in blocking buffer) for two hours at RT. After several washings with PBS-T, wells are incubated with 100 μl of 1:2000 dilution of HRP-conjugated anti-human IgG in blocking buffer for one hour. After washing, 100 μl TMB-developing solution is added to each well, which is then incubated at RT. The reaction is stopped by the addition of 1 M H₂SO₄ and read at 450 nm. For negative controls, wells are not incubated with sample, and other wells are not coated with antigen but are incubated with sample. For negative control values, the mean of both negative control values are presented.

### ELISA on Purified Recombinant Proteins

ELISA experiments were performed as described for peptides, except that 96-well ELISA plates (Greiner) were coated with 100 μl of 1 μg/ml purified proteins (UH-CSFP1.3, UH-CSFP1.6, UH-CSFP1.7 and UH-CSFP1.8) in coating buffer and kept overnight at 4° C. Serum samples were considered positive for antibodies against the purified proteins when the OD₄₅₀ was higher than the mean+3 times the standard deviation of the healthy controls. The horizontal line in FIG. 8 represents the cut-off value.

### SPAG16 ELISA

96-well ELISA plates (Greiner Bio-One) were coated with 1 μg/ml purified recombinant protein in 0.1 M bicarbonate buffer (pH 9.6) and kept overnight at 4° C. Washing was done using 0.05% PBS-TWEEN®20 (PBS-T). Wells were blocked with 2% M-PBS for two hours at 37° C. The plates were incubated with 100 μl of diluted plasma samples (1:100 in M-PBS) for two hours at room temperature (RT). Detection of antibody binding was performed with HRP-conjugated anti-human IgG (1:2000 in M-PBS; Dako) followed by color development with TMB-solution (Sigma-Aldrich). The reaction was stopped with 2 M H₂SO₄ and read at 450 nm. Background reactivity was accounted for by measuring in parallel immunoreactivity against a recombinant THIO protein. The Coefficient of Variation (% CV) was on average ˜5% and had to be <20%, otherwise an experiment was repeated. A serial dilution of a positive sample was included in each experiment to test for interassay variability. The cut-off was determined by ROC-analysis.

To analyze the IgG isotype of the anti-SPAG16 antibodies, SPAG16 protein ELISA assays were performed as mentioned above with a representative number of positive plasma samples of MS patients (n=22). An isotype specific secondary antibody was used for detection; anti-IgG1, anti-IgG2, anti-IgG3 (all from Invitrogen) and anti-IgG4 (AbD Serotec), respectively.

### Isoelectric Focusing (IEF)

Recombinant SPAG16-2 or Thioredoxin (control) was expressed and purified according to the pBAD/TOPOThioFusion kit (Invitrogen). Protein purity was assessed by SDS-PAGE and Coomassie Brilliant Blue (Phastgel Blue-R, GE Healthcare). Protein identity was conformed by ESI-LC-MS/MS analysis (ThermoFinnigan). Protein concentration was determined with the BCA protein quantification kit (Thermo Fisher Scientific). To investigate whether oligoclonal IgG recognizes SPAG16, three sets of CSF samples of four MS patients were subjected to IEF to separate IgG, as previously described.¹⁷ Briefly, nitrocellulose membranes (Amersham Biosciences) were incubated overnight (4° C.; shaking), with either SPAG16 or thioredoxin, both 20 μg/ml in PBS. A third membrane was not coated. After incubation, the membranes were washed in 0.2% non-fat milk in PBS (M-PBS) and blocked 18 hours at 4° C. in 2% M-PBS. Before use, they were rinsed in PBS. After IEF, proteins were transferred to the three nitrocellulose membranes. The presence of oligoclonal IgG bands linked to the membranes was investigated by immunoblotting with a phosphatase alkaline anti-human IgG antibody (Jackson ImmunoResearch Laboratories Inc.) as previously described.⁵³ Investigation of oligoclonal band reactivity was explored by a modification of a previously described method.⁵⁴

### Production and Specificity of Mouse Monoclonal Anti-SPAG16 Antibodies.

Mouse monoclonal antibodies (mAbs) were produced against recombinant SPAG16-2 using the hybridoma technology.⁵⁵ Antibodies were purified from tissue culture supernatant by POROS A (Applied Biosystems) affinity-chromatography for IgG and Protein L (Thermo Fisher Scientific) chromatography for IgM. Eluted antibodies were dialyzed (Thermo Fisher Scientific) against PBS. Purity of the produced mAbs was analyzed by SDS-PAGE and concentrations were determined with the BCA kit (Thermo Fisher Scientific). Isotypes of the produced antibodies were determined with the mouse monoclonal antibody isotyping kit according to the manufacturer's recommendations (Hycult Biotechnology). The specificity of the produced mAbs was confirmed by western blot and by the application of competition protein ELISA with antibody-positive MS plasma samples. They bind both full size SPAG16 and the 121 as fragment.

### Immunohistochemistry (IHC).

Formalin-fixed paraffin-embedded or frozen tissue sections from MS (n=7) and non-demented control (n=5) brain tissue were used (Dutch Brain Bank). Paraffin-embedded sections were deparaffinized and rehydrated. Endogenous peroxidase activity was inhibited using 0.3% hydrogen peroxide in methanol and nonspecific antibody binding was blocked with 10% rabbit serum in PBS or Protein Block (Dako). The sections were incubated overnight at 4° C. with different primary antibodies (see Table 8). Bound antibodies were detected with the Dako-Envision detection system or HRP-conjugated secondary antibodies (Dako) followed by color development with DAB (Dako). Counterstaining was performed with hematoxylin (Klinipath). An identically produced isotype control mAb was used as a negative control.

Human and murine frozen tissue sections (5 μm) were fixed in aceton for ten minutes at RT and were then washed in PBS-T. After blocking for 30 minutes at RT with Protein Block (Dako), sections were incubated overnight at 4° C. or one hour at RT with primary antibodies diluted in PBS containing 0.1% BSA (see Table 8). After washing with PBS-T, fluorescent secondary antibodies (Invitrogen) were added for one hour at RT. Cell nuclei were fluorescently labelled with DAPI. Sections were incubated for ten minutes in 0.1% Sudan Black (Sigma-Aldrich) in 70% ethanol to block endogenous autofluorescence. Stained tissue sections were evaluated with an Eclipse 80i microscope (Nikon), using standard objectives and NIS-Elements Basic Research Software (Nikon).

### Western Blotting of Mouse Spinal Cord Lysates.

Normal mouse spinal cord tissues were homogenized with a rotor-stator in lysis buffer (150 mM sodium chloride, 1% (v/v) NP-40 (Nonidet P-40, Sigma-Aldrich), 50 mM Tris, pH 8.0) containing EDTA-free nuclease inhibitors (Roche Diagnostics) followed by clearing the lysate by centrifugation. The protein concentration of the supernatant was determined with the BCA kit (Thermo Fisher Scientific). Twenty μg of spinal cord lysate proteins were separated by 12% SDS-PAGE and blotted onto a PVDF (polyvinylidene fluoride)-membrane (Millipore). The membrane was blocked (one hour in 5% in MPBS-T) and incubated with mAbs (30 μg/ml) in 2% MPBS-T overnight at 4° C. Bound antibodies were detected with secondary rabbit anti-mouse IgG-HRP (1:800, Dako) for one hour at RT, followed by color development by DAB substrate (Sigma-Aldrich) or chemiluminescence (Thermo Fisher Scientific). A monoclonal mouse isotype antibody (IgG1) directed against an irrelevant protein was used as a negative control.

### EAE Induction and Passive Antibody Transfer.

Female C57BL/6J mice were purchased from Harlan Netherlands B.V., were kept at standard laboratory conditions and were fed at libitum. Experiments were conducted in accordance with institutional guidelines and were approved by the ethical committee for animal experiments of Hasselt University. To induce EAE, animals were immunized subcutaneously with 100 μl PBS containing 200 μg MOG³⁵⁻⁵⁵ peptide (Ansynth) and 100 μl CFA (Sigma) supplemented with 500 μg mycobacterium tuberculosis (Sigma). Directly after immunization and 48 hours later, animals were injected i.p. with 200 ng pertussis toxin (Sigma). Animals were weighed and scored daily for clinical signs of EAE. Clinical disease was graded using a standard five-point EAE scale with increments of 0.5 points. At EAE onset (score 0.5-1), animals were injected i.p. with 2 mg of the anti-SPAG16 or isotype control antibody mix. The anti-SPAG16 antibody mix was composed of three different mAbs (3H5 (IgM), 1F1 (IgG1) and 5F10 (IgG1)), each constituting one-third of the total injected antibody amount. The isotype control antibody mix contained ⅔ anti-human chorion gonadotrophin IgG1 and ⅓ anti-treponema pallidum IgM. Scoring was done blinded for experimental design. Mice were sacrificed at day 17 post-antibody transfer (˜day 30 after disease induction). Histological quantification was performed on sections obtained at three different spinal cord levels in each animal (Eclipse 80i microscope with NIS-Elements Basic Research Software (Nikon)).

### Statistical Analysis

Statistical analysis for Examples 1-10 was performed using GraphPad Prism version 4.0. Quantitative demographic variables for antibody-positive and antibody-negative individuals were compared using t-tests, and categorical variables were compared using chi-square tests. A p value <0.05 was considered statistically significant. Correlations between various markers were determined by linear regression analysis.

Statistical analysis for Examples 11-13 was performed using GraphPad Prism (version 5.0, GraphPad Software Inc.) and SPSS statistical software (version 20). A P-value <0.05 was considered statistically significant. Specific tests and significance levels are described in figure legends.

TABLE 1
Characteristics of the study population

| | | | Mean age (SD) |
|---|---|---|---|
| Diagnosis | No. | Female/Male | (range) in years |
| MS | 73 | 51/22 | 38.6 (9.5) (16-57) |
| RR-MS | 64 | | 37.4 (9.0) (16-56) |
| SP-MS | 3 | | 48.7 (11) (36-56) |
| PP-MS | 6 | | 47.3 (8.3) (38-57) |
| NIND | 64 | 34/30 | 55.4 (17.6) (21-93) |
| OIND | 30 | 15/15 | 43.5 (15) (19-81) |

TABLE 2
Characteristics of patients used for affinity selections

| | | | Age start | Disease | | |
|---|---|---|---|---|---|---|
| | Gender | Age | disease | duration | | |
| Subject | (M/F) | (years) | (years) | (years) | Diagnosis | EDSS |
| 1 | F | 49 | 40 | 8 | RR-MS | 3.5 |
| 2 | M | 42 | 34 | 8 | RR-MS | 1.5 |
| 3 | F | 52 | 49 | 3 | RR-MS | 1.5 |
| 4 | F | 41 | 38 | 3 | RR-MS | 3.0 |
| 5 | F | 52 | 51 | 0.5 | RR-MS | 1.0 |
| 6 | F | 48 | 46 | 0.6 | RR-MS | 1.5 |
| 7 | F | 46 | 44 | 1 | RR-MS | 2.5 |
| 8 | F | 43 | 35 | 7 | RR-MS | 1.5 |
| 9 | F | 36 | 32 | 3 | RR-MS | 1.5 |
| 10 | M | 31 | 27 | 4 | RR-MS | 0 |

TABLE 3
Sequence analysis of antigens retrieved by the selection of phage
displayed MS cDNA expression library on MS patient CSF
Antigens

| | | | | Translated | | | SEQ |
|---|---|---|---|---|---|---|---|
| | GenBank | Vector | | amino acid | | | ID |
| Name | No. | pSP^{a} | Identity | sequence^{b} | Size^{c} | Comments | NO: |
| UH-CSFP1.1 | NM014382 | B | HS ATP2C1, transcript | ASSRGYEDLR | 12 | 3′ UTR | 1 |
| | | | variant 1 | | | | |
| UH-CSFP1.2 | NM199478 | B | PLP1, transcript | LDNSYHDNPV | 23 | 3′ UTR | 2 |
| | | | variant 2 | | | | |
| UH-CSFP1.3 | BX509701.1 | A | HS DKFZp686A1481 | LRAPAGLGAA | 52 | Est | 3 |
| UH-CSFP1.4 | BC006427 | C | HS KIAA1279 | GARCINAEQP | 14 | out of | 4 |
| | | | | | | frame | |
| UH-CSFP1.5 | NM00729 | B | HS PACSIN2 | YSCLKLYSFA | 11 | 3′ UTR | 5 |
| UH-CSFP1.6 | AC114947.2 | A | HS chromosome 5 | EHATQNQVSV | 103 | | 6 |
| | | | clone CTD-2636A23 | | | | |
| UH-CSFP1.7 | BC032450 | A | HS chromosome 10 ORF | GTGSGQGEEA | 54 | | 7 |
| | | | with retained intron | | | | |
| UH-CSFP1.8 | BC067756.1 | A | HS sperm associated | ADDNFSIPEG | 121 | in-frame | 8 |
| | | | antigen 16 | | | | |

^{a}Reading frame of vector
^{b}Translated amino acid sequence of the cDNA insert according to the reading frame of the vector: the first ten aa of the fusion product are presented with SEQ ID NOs indicated at the right.
^{c}Size of protein product in amino acids fused to pVI coat protein, * stop codon

TABLE 4
Reactivity of panel of eight phage clones on individual MS CSF used for the selection procedure

| | | CSF | | | | | | | | | | | |
|---|---|---|---|---|---|---|---|---|---|---|---|---|---|
| | | CSF1 | CSF2 | CSF3 | CSF4 | CSF5 | CSF6 | CSF7 | CSF8 | CSF9 | CSF10 | | |
| Phage | UH- | − | − | − | + | − | − | + | + | − | − | 3/10 | Number |
| cDNA | CSFP1.1 | | | | | | | | | | | | of |
| clones | UH- | − | − | − | + | + | − | − | + | − | + | 4/10 | reactive |
| | CSFP1.2 | | | | | | | | | | | | CSF |
| | UH- | − | − | − | − | − | − | − | − | + | − | 1/10 | |
| | CSFP1.3 | | | | | | | | | | | | |
| | UH- | + | + | − | − | − | − | − | − | − | + | 3/10 | |
| | CSFP1.4 | | | | | | | | | | | | |
| | UH- | + | + | − | − | − | − | − | − | + | + | 4/10 | |
| | CSFP1.5 | | | | | | | | | | | | |
| | UH- | + | + | − | − | + | − | + | − | + | + | 6/10 | |
| | CSFP1.6 | | | | | | | | | | | | |
| | UH- | − | − | − | − | − | − | − | − | − | + | 1/10 | |
| | CSFP1.7 | | | | | | | | | | | | |
| | UH- | − | − | − | − | − | − | − | − | − | + | 1/10 | |
| | CSFP1.8 | | | | | | | | | | | | |
| | Number of positive | 3/8 | 3/8 | 0/8 | 2/8 | 2/8 | 0/8 | 2/8 | 2/8 | 3/8 | 6/8 | | |
| | phage cDNA clones | | | | | | | | | | | | |

+ positive ELISA signal at OD450 nm (>1.5x background)
− negative ELISA signal at OD450 nm (<1.5x background)

TABLE 5
ELISA screening of individual phage-
cDNA clones on 167 different CSF

| | | | | | | Total |
|---|---|---|---|---|---|---|
| | Selec- | Non- | | | | Con- |
| Name | tion^{a} | Selection^{b} | Total | NIND^{c} | OIND^{d} | trols |
| UH-CSFP1.1 | 3/10 | 3/63 | 6/73 | 3/64 | 1/30 | 4/94 |
| UH-CSFP1.2 | 4/10 | 6/63 | 10/73 | 1/64 | 0/30 | 1/94 |
| UH-CSFP1.3 | 1/10 | 4/63 | 5/73 | 0/64 | 1/30 | 1/94 |
| UH-CSFP1.4 | 3/10 | 1/63 | 4/73 | 0/64 | 0/30 | 0/94 |
| UH-CSFP1.5 | 4/10 | 2/63 | 6/73 | 0/64 | 0/30 | 0/94 |
| UH-CSFP1.6 | 6/10 | 4/63 | 10/73 | 0/64 | 0/30 | 0/94 |
| UH-CSFP1.7 | 1/10 | 4/63 | 5/73 | 0/64 | 0/30 | 0/94 |
| UH-CSFP1.8 | 1/10 | 13/63 | 14/73 | 7/64 | 0/30 | 7/94 |

^{a}individual antigen reactive CSF from MS patients used in the selection procedure
^{b}individual CSF from MS patients not used in the selection procedure
^{c}NIND: hernia, epilepsy, dementia, headache, migraine, Alzheimer, hydrocephalus
^{d}OIND: meningitis, polyneuropathy

TABLE 6
Comparison of antibody-positive and antibody-
negative patients with established MS

| | Antibody | Antibody | |
|---|---|---|---|
| | Positive | Negative | |
| Characteristic | (n = 33) | (n = 40) | P |
| Age, mean ± SD years | 37.7 ± 8.9 | 39.4 ± 10.0 | NS |
| Disease duration, mean ± SD years | 3.6 ± 3.3 | 4.3 ± 5.2 | NS |
| Sex | | | |
| Male | 11 | 11 | |
| Female | 22 | 29 | |
| EDSS, gem ± SD | 2 ± 1 | 2 ± 1 | NS |

Age and disease duration were compared by t-test, and categorical variables were compared by chi-square testing with appropriate degrees of freedom. NS = not significant

TABLE 7
Characteristics of the MS patients and
controls used in the extended cohort

| | | | | Mean | Mean disease |
|---|---|---|---|---|---|
| Diagnosis | Number | Mean age^{a} | Gender^{b} | EDSS^{c} | duration^{d} |
| MS | 153 | 46.4 ± 0.9 | 67% | 3.8 ± 0.2 | 11.7 ± 0.8 |
| RRMS | 70 | 42.8 ± 1.1 | 74% | 2.7 ± 0.2 | 10.6 ± 1.4 |
| PPMS | 25 | 51. ± 2.1 | 52% | 4.1 ± 0.4 | 8.1 ± 1.3 |
| SPMS | 58 | 49.3 ± 1.4 | 64% | 4.9 ± 0.3 | 15.2 ± 1.2 |
| CIS^{e} | 101 | 35.5 ± 1.0 | 79% | 1.6 ± 0.1 | 0.4 ± 0.1 |
| Healthy | 204 | 39.5 ± 1.8 | 65% | N.A. | N.A. |
| controls | | | | | |
| NIND^{f} | 51 | 43.8 ± 2.6 | 73% | N.A. | N.A. |
| OIND^{g} | 22 | 46.7 ± 3.1 | 59% | N.A. | N.A. |

^{a}Mean age in years ± SEM
^{b}% females
^{c}Mean EDSS (Expanded Disability Status Scale) score ± SEM
^{d}Mean disease duration in years ± SEM
^{e}CIS = clinically isolated syndrome
^{f}NIND = non-inflammatory neurological disease
^{g}OIND = other inflammatory neurological disease
N.A. not applicable

TABLE 8
Primary Abs used in this study

| | | Concentration/ | |
|---|---|---|---|
| Primary Abs | Specificity | dilution | Source |
| 1F1 and 5F10 | SPAG16 | 30 μg/ml | In-house |
| | | | produced |
| Isotype control | hCG | 30 μg/ml | In-house |
| | | | produced |
| IE7 isotype | Rubella | 30 μg/ml | In-house |
| control | | | produced |
| MAB386 | MBP | 1:100 | Millipore |
| CR3/43 | HLA- | 1:100 | Dako |
| | DR, DP, DQ | | |
| EB09826 | SPAG16 | 1:200 | Everest Biotech |
| Clone G-A-5 | GFAP | 1:500 | Sigma-Aldrich |
| Clone A60 | NeuN | 1:200 | Millipore |
| CD3-12 | CD3 | 1:100 | Serotec |
| clone Cl:A3-1 | F4/80 | 1:500 | Serotec |

hCG, human chorionic gonadotropin; MBP, myelin basic protein; GFAP, glial fibrillary acidic protein; NeuN, neuronal nuclei

TABLE 9
Clinical features of EAE after passive transfer
of anti-SPAG16 or isotype control antibodies

| | Disease | Maximum | Cumulative | Days with |
|---|---|---|---|---|
| | Score^{a} | score^{b} | score^{c} | score ≧2^{d} |
| SPAG16 | 2.1** | 4.0 | 39.6 ± 2.8** | 14.8 ± 1.5** |
| Isotype | 1.3 | 2.3 | 25.1 ± 2.9 | 5.5 ± 1.3 |

^{a}Median disease score over the period of follow-up (day 1-17 post-antibody transfer); repeated measures ANOVA.
^{b}Median of the maximum scores; Mann-Whitney U testing.
^{c}Mean of the cumulative scores of all animals in one group. Cumulative score per animal is the sum of the daily clinical scores during follow-up. Groups were compared by Student T-test.
^{d}Mean number of follow-up days with disease score of at least two for all animals in one group. Groups were compared with Student T-test.
*<0.05;
**p < 0.01

### REFERENCES

1. Lucchinetti C. F., W. Bruck, M. Rodriguez, and H. Lassmann. Distinct patterns of multiple sclerosis pathology indicates heterogeneity on pathogenesis. Brain Pathol. 6:259-274, 1996.
2. Lucchinetti C., W. Bruck, J. Parisi, B. Scheithauer, M. Rodriguez, and H. Lassmann. Heterogeneity of multiple sclerosis lesions: implications for the pathogenesis of demyelination. Ann. Neurol. 47:707-717, 2000.
3. Genain C. P., B. Cannella, S. L. Hauser, and C. S. Raine. Identification of autoantibodies associated with myelin damage in multiple sclerosis. Nat. Med. 5:170-175, 1999.
4. Cross A. H., J. L. Trotter, and J. Lyons. B cells and antibodies in CNS demyelinating disease. J. Neuroimmunol. 112:1-14, 2001.
5. Qin Y., P. Duquette, Y. Zhang, P. Talbot, R. Poole, and J. Antel. Clonal expansion and somatic hypermutation of V(H) genes of B cells from cerebrospinal fluid in multiple sclerosis. J. Clin. Invest. 102:1045-1050, 1998.
6. Baranzini S. E., M. C. Jeong, C. Butunoi, R. S. Murray, C. C. Bernard, and J. R. Oksenberg. B cell repertoire diversity and clonal expansion in multiple sclerosis brain lesions. J. Immunol. 163:5133-5144, 1999.
7. Karni A., R. Bakimer-Kleiner, O. Abramsky, and A. Ben Nun. Elevated levels of antibody to myelin oligodendrocyte glycoprotein is not specific for patients with multiple sclerosis. Arch. Neurol. 56:311-315, 1999.
8. Moller J. R., D. Johnson, R. O. Brady, W. W. Tourtellotte, and R. H. Quarles. Antibodies to myelin-associated glycoprotein (MAG) in the cerebrospinal fluid of multiple sclerosis patients. J. Neuroimmunol. 22:55-61, 1989.
9. Vojdani A., E. Vojdani, and E. Cooper. Antibodies to myelin basic protein, myelin oligodendrocytes peptides, alpha-beta-crystallin, lymphocyte activation and cytokine production in patients with multiple sclerosis. J. Intern. Med. 254:363-374, 2003.
10. Lalive P. H., T. Menge, C. Delarasse, G. B. Della, D. Pham-Dinh, P. Villoslada, H. C. von Budingen, and C. P. Genain. Antibodies to native myelin oligodendrocyte glycoprotein are serologic markers of early inflammation in multiple sclerosis. Proc. Natl. Acad. Sci. U.S.A. 103:2280-2285, 2006.
11. O'Connor K. C., T. Chitnis, D. E. Griffin, S. Piyasirisilp, A. Bar-Or, S. Khoury, K. W. Wucherpfennig, and D. A. Hafler. Myelin basic protein-reactive autoantibodies in the serum and cerebrospinal fluid of multiple sclerosis patients are characterized by low-affinity interactions. J. Neuroimmunol. 136:140-148, 2003.
12. Almeras L., D. Lefranc, H. Drobecq, J. de Seze, S. Dubucquoi, P. Vermersch, and L. Prin. New antigenic candidates in multiple sclerosis: identification by serological proteome analysis. Proteomics. 4:2184-2194, 2004.
13. Kolln J., H. M. Ren, R. R. Da, Y. Zhang, E. Spillner, M. Olek, N. Hermanowicz, L. G. Hilgenberg, M. A. Smith, N. S. Van Den, and Y. Qin. Triosephosphate isomerase- and glyceraldehyde-3-phosphate dehydrogenase-reactive autoantibodies in the cerebrospinal fluid of patients with multiple sclerosis. J. Immunol. 177:5652-5658, 2006.
14. Poser C. M., D. W. Paty, L. Scheinberg, W. I. McDonald, F. A. Davis, G. C. Ebers, K. P. Johnson, W. A. Sibley, D. H. Silberberg, and W. W. Tourtellotte. New diagnostic criteria for multiple sclerosis: guidelines for research protocols. Ann. Neurol. 13:227-231, 1983.
15. Somers V., C. Govarts, N. Hellings, R. Hupperts, and P. Stinissen. Profiling the autoantibody repertoire by serological antigen selection. J. Autoimmun. 25:223-228, 2005.
16. Marks J. D., H. R. Hoogenboom, T. P. Bonnert, J. McCafferty, A. D. Griffiths, and G. Winter. By-passing immunization. Human antibodies from V-gene libraries displayed on phage. J. Mol. Biol. 222:581-97, 1991.
17. Somers V. A., R. J. Brandwijk, B. Joosten, P. T. Moerkerk, J. W. Arends, P. Menheere, W. O. Pieterse, A. Claessen, R. J. Scheper, H. R. Hoogenboom, and S. E. Hufton. A panel of candidate tumor antigens in colorectal cancer revealed by the serological selection of a phage displayed cDNA expression library. J. Immunol. 169:2772-2780, 2002.
18. Cortese I., R. Tafi, L. M. Grimaldi, G. Martino, A. Nicosia, and R. Cortese. Identification of peptides specific for cerebrospinal fluid antibodies in multiple sclerosis by using phage libraries. Proc. Natl. Acad. Sci. U.S.A, 93:11063-11067, 1996.
19. Archelos J. J., J. Trotter, S. Previtali, B. Weissbrich, K. V. Toyka, and H. P. Hartung. Isolation and characterization of an oligodendrocyte precursor-derived B-cell epitope in multiple sclerosis. Ann. Neurol., 43:15-24, 1998.
20. Owens G. P., M. P. Burgoon, M. E. Devlin, and D. H. Gilden. Strategies to identify sequences or antigens unique to multiple sclerosis. Mult. Scler., 2:184-194, 1996.
21. Cortese I., S. Capone, S. Luchetti, L. M. Grimaldi, A. Nicosia, and R. Cortese. CSF-enriched antibodies do not share specificities among MS patients. Mult. Scler., 4:118-123, 1998.
22. Becker K. G., D. H. Mattson, J. M. Powers, A. M. Gado, and W. E. Biddison. Analysis of a sequenced cDNA library from multiple sclerosis lesions. J. Neuroimmunol. 77:27-38, 1997.
23. Robinson W. H., P. Fontoura, B. J. Lee, H. E. de Vegvar, J. Tom, R. Pedotti, C. D. DiGennaro, D. J. Mitchell, D. Fong, P. P. Ho, P. J. Ruiz, E. Maverakis, D. B. Stevens, C. C. Bernard, R. Martin, V. K. Kuchroo, J. M. ort, C. P. Genain, S. Amor, T. Olsson, P. J. Utz, H. Garren, and L. Steinman. Protein microarrays guide tolerizing DNA vaccine treatment of autoimmune encephalomyelitis. Nat. Biotechnol. 21:1033-1039, 2003.
24. Neer E. J., C. J. Schmidt, R. Nambudripad, and T. F. Smith. The ancient regulatory-protein family of WD-repeat proteins. Nature 371:297-300, 1994.
25. Smith T. F., C. Gaitatzes, K. Saxena and E. J. Neer. The WD repeat: a common architecture for diverse functions. Trends Biochem. Sci. 24:181-185, 1999.
26. Smith E. F. and P. A. Lefebvre. PF20 gene product contains WD repeats and localizes to the intermicrotubule bridges in Chlamydomonas flagella. Mol. Biol. Cell 8:455-467, 1997.
27. Strausberg R. L., E. A. Feingold, L. H. Grouse, J. G. Derge, R. D. Klausner, F. S. Collins, L. Wagner, C. M. Shenmen, G. D. Schuler, S. F. Altschul, B. Zeeberg, K. H. Buetow, C. F. Schaefer, N. K. Bhat, R. F. Hopkins, H. Jordan, T. Moore, S. I. Max, J. Wang, F. Hsieh, L. Diatchenko, K. Marusina, A. A. Farmer, G. M. Rubin, L. Hong, M. Stapleton, M. B. Soares, M. F. Bonaldo, T. L. Casavant, T. E. Scheetz, M. J. Brownstein, T. B. Usdin, S. Toshiyuki, P. Carninci, C. Prange, S. S. Raha, N. A. Loquellano, G. J. Peters, R. D. Abramson, S. J. Mullahy, S. A. Bosak, P. J. McEwan, K. J. McKernan, J. A. Malek, P. H. Gunaratne, S. Richards, K. C. Worley, S. Hale, A. M. Garcia, L. J. Gay, S. W. Hulyk, D. K. Villalon, D. M. Muzny, E. J. Sodergren, X. Lu, R. A. Gibbs, J. Fahey, E. Helton, M. Ketteman, A. Madan, S. Rodrigues, A. Sanchez, M. Whiting, M., A. Madan, A. C. Young, Y. Shevchenko, G. G. Bouffard, R. W. Blakesley, J. W. Touchman, E. D. Green, M. C. Dickson, A. C. Rodriguez, J. Grimwood, J. Schmutz, R. M. Myers, Y. S. Butterfield, M. I. Krzywinski, U. Skalska, D. E. Smailus, A. Schnerch, J. E. Schein, S. J. Jones, and M. A. Marra. Generation and initial analysis of more than 15,000 full-length human and mouse cDNA sequences. Proc. Natl. Acad. Sci. U.S.A. 99:16899-16903, 2002.
28. Ota T., Y. Suzuki, T. Nishikawa, T. Otsuki, T. Sugiyama, R. Irie, A. Wakamatsu, K. Hayashi, H. Sato, K. Nagai, K. Kimura, H. Makita, M. Sekine, M. Obayashi, T. Nishi, T. Shibahara, T. Tanaka, S. Ishii, J. Yamamoto, K. Saito, Y. Kawai, Y. Isono, Y. Nakamura, K. Nagahari, K. Murakami, T. Yasuda, T. Iwayanagi, M. Wagatsuma, A. Shiratori, H. Sudo, T. Hosoiri, Y. Kaku, H. Kodaira, H. Kondo, M. Sugawara, M. Takahashi, K. Kanda, T. Yokoi, T. Furuya, E Kikkawa, Y. Omura, K. Abe, K. Kamihara, N. Katsuta, K. Sato, M. Tanikawa, M. Yamazaki, K. Ninomiya, T. Ishibashi, H. Yamashita, K. Murakawa, K. Fujimori, H. Tanai, M. Kimata, M. Watanabe, S. Hiraoka, Y. Chiba, S. Ishida, Y. Ono, S. Takiguchi, S. Watanabe, M. Yosida, T. Hotuta, J. Kusano, K. Kanehori, A. Takahashi-Fujii, H. Hara, T. O. Tanase, Y. Nomura, S. Togiya, F. Komai, R. Hara, K. Takeuchi, M. Arita, N. Imose, K. Musashino, H. Yuuki, A. Oshima, N. Sasaki, S. Aotsuka, Y. Yoshikawa, H. Matsunawa, T. Ichihara, N. Shiohata, S. Sano, S. Moriya, H. Momiyama, N. Satoh, S. Takami, Y. Terashima, O. Suzuki, S. Nakagawa, A. Senoh, H. Mizoguchi, Y. Goto, F. Shimizu, H. Wakebe, H. Hishigaki, T. Watanabe, A. Sugiyama, M. Takemoto, B. Kawakami, M. Yamazaki, K. Watanabe, A. Kumagai, S. Itakura, Y. Fukuzumi, Y. Fujimori, M. Komiyama, H. Tashiro, A. Tanigami, T. Fujiwara, T. Ono, K. Yamada, Y. Fujii, K. Ozaki, M. Hirao, Y. Ohmori, A. Kawabata, T. Hikiji, N. Kobatake, H. Inagaki, Y. Ikema, S. Okamoto, R. Okitani, T. Kawakami, S. Noguchi, T. Itoh, K. Shigeta, T. Senba, K. Matsumura, Y. Nakajima, T. Mizuno, M. Morinaga, M. Sasaki, T. Togashi, M. Oyama, H. Hata, M. Watanabe, T. Komatsu, J. Mizushima-Sugano, T. Satoh, Y. Shirai, Y. Takahashi, K. Nakagawa, K. Okumura, T. Nagase, N. Nomura, H. Kikuchi, Y. Masuho, R. Yamashita, K. Nakai, T. Yada, Y. Nakamura, O. Ohara, T. Isogai, and S. Sugano. Complete sequencing and characterization of 21,243 full-length human cDNAs. Nat. Genet. 36:40-45, 2004.
29. Ng B., F. Yang, D. P. Huston, Y. Yan, Y. Yang, Z. Xiong, L. E. Peterson, H. Wang, and X. F. Yang. Increased noncanonical splicing of autoantigen transcripts provides the structural basis for expression of untolerized epitopes. J. Allergy Clin. Immunol. 114:1463-1470, 2004.
30. Yeo G., D. Holste, G. Kreiman, and C. B. Burge. Variation in alternative splicing across human tissues. Genome Biol. 5:R74, 2004.
31. Larsen J. E., O. Lund, and M. Nielsen. Improved method for predicting linear B-cell epitopes. Immunome. Res. 2:2, 2006.
32. Cepok S., D. Zhou, R. Srivastava, S. Nessler, S. Stei, K. Bussow, N. Sommer, and B. Hemmer. Identification of Epstein-Barr virus proteins as putative targets of the immune response in multiple sclerosis. J. Clin. Invest. 115:1352-1360, 2005.
33. Wang X., J. Yu, A. Sreekumar, S. Varambally, R. Shen, D. Giacherio, R. Mehra, J. E. Montie, K. J. Pienta, M. G. Sanda, P. W. Kantoff, M. A. Rubin, J. T. Wei, D. Ghosh, and A. M. Chinnaiyan. Autoantibody signatures in prostate cancer. N Engl. J. Med. 353:1224-1235, 2005.
34. Keegan M., F. Konig, R. McClelland, W. Bruck, Y. Morales, A. Bitsch, H. Panitch, H. Lassmann, B. Weinshenker, M. Rodriguez, J. Parisi, and C. F. Lucchinetti. Relation between humoral pathological changes in multiple sclerosis and response to therapeutic plasma exchange. Lancet 366:579-582, 2005.
35. Stuve O., S. Cepok, B. Elias, A. Saleh, H. P. Hartung, B. Hemmer, and B. C. Kieseier. Clinical stabilization and effective B-lymphocyte depletion in the cerebrospinal fluid and peripheral blood of a patient with fulminant relapsing-remitting multiple sclerosis. Arch. Neurol. 62:1620-1623, 2005.
36. Zhang Z., M. A. Zariwala, M. M. Mahadevan et al. A heterozygous mutation disrupting the SPAG16 gene results in biochemical instability of central apparatus components of the human sperm axoneme. Biology of reproduction. 2007 November; 77(5):864-71.
37. Pennarun G., A. M. Bridoux, and E. Escudier, et al. Isolation and expression of the human hPF20 gene orthologous to Chlamydomonas PF20: evaluation as a candidate for axonemal defects of respiratory cilia and sperm flagella. American journal of respiratory cell and molecular biology 2002 March; 26 (3):362-70.
38. Silina K., P. Zayakin, Z. Kalnina, et al. Sperm associated antigens as targets for cancer immunotherapy: expression pattern and humoral immune response in cancer patients. Journal of immunotherapy 2011 January; 34(1):28-44.
39. Uhlen M., E. Bjorling, C. Agaton, et al. A human protein atlas for normal and cancer tissues based on antibody proteomics. Molecular & cellular proteomics: MCP. 2005 December; 4(12):1920-32.
40. Knevel R., K. Klein, K. Somers, et al. Identification of a genetic variant for joint damage progression in autoantibody-positive rheumatoid arthritis. Annals of the rheumatic diseases 2013 Aug. 16.
41. Awad A., B. Hemmer, H. P. Hartung, B. Kieseier, J. L. Bennett, O. Stuve. Analyses of cerebrospinal fluid in the diagnosis and monitoring of multiple sclerosis. Journal of neuroimmunology 2010 Feb. 26; 219(1-2):1-7.
42. Hawker K., P. O'Connor, M. S. Freedman, et al. Rituximab in patients with primary progressive multiple sclerosis: results of a randomized double-blind placebo-controlled multicenter trial. Annals of neurology 2009 October; 66(4):460-71.
43. Eikelenboom M. J., A. Petzold, R. H. Lazeron, et al. Multiple sclerosis: Neurofilament light chain antibodies are correlated to cerebral atrophy. Neurology. 2003 Jan. 28; 60(2):219-23.
44. Svarcova J., L. Fialova, A. Bartos, M. Steinbachova, I. Malbohan. Cerebrospinal fluid antibodies to tubulin are elevated in the patients with multiple sclerosis. European journal of neurology. The official journal of the European Federation of Neurological Societies 2008 November; 15(11):1173-9.
45. Racanelli V., M. Prete, G. Musaraj, F. Dammacco, F Perosa. Autoantibodies to intracellular antigens: generation and pathogenetic role. Autoimmunity reviews 2011 June; 10(8):503-8.
46. Yasuoka H., H. Ihn, T. A. Medsger, Jr., et al., A novel protein highly expressed in testis is overexpressed in systemic sclerosis fibroblasts and targeted by autoantibodies. Journal of immunology 2003 Dec. 15; 171(12):6883-90.
47. van Noort J. M., A. C. van Sechel, J. J. Bajramovic, et al. The small heat-shock protein alpha B-crystallin as candidate autoantigen in multiple sclerosis. Nature 1995 Jun. 29; 375(6534):798-801.
48. Zhou D., R. Srivastava, S. Nessler, et al. Identification of a pathogenic antibody response to native myelin oligodendrocyte glycoprotein in multiple sclerosis. Proceedings of the National Academy of Sciences of the United States of America 2006 Dec. 12; 103(50):19057-62.
49. Mayer M. C., E. Meinl. Glycoproteins as targets of autoantibodies in CNS inflammation: MOG and more. Therapeutic advances in neurological disorders 2012 May; 5(3):147-59.
50. Mathey E. K., T. Derfuss, M. K. Storch, et al. Neurofascin as a novel target for autoantibody-mediated axonal injury. The Journal of experimental medicine 2007 Oct. 1; 204(10):2363-72.
51. Kawamura N., R. Yamasaki, T. Yonekawa, et al. Anti-neurofascin antibody in patients with combined central and peripheral demyelination. Neurology 2013 Aug. 20; 81(8):714-22.
52. McDonald W. I., A. Compston, G. Edan, et al. Recommended diagnostic criteria for multiple sclerosis: guidelines from the International Panel on the diagnosis of multiple sclerosis. Annals of neurology 2001 July; 50(1):121-7.
53. Sadaba M. C., P. Gonzalez Porque, J. Masjuan, J. C. Alvarez-Cermeno, A. Bootello, L. M. Villar. An ultrasensitive method for the detection of oligoclonal IgG bands. Journal of immunological methods 2004 January; 284(1-2):141-5.
54. Villar L. M., M. C. Sadaba, E. Roldan, et al. Intrathecal synthesis of oligoclonal IgM against myelin lipids predicts an aggressive disease course in MS. The Journal of clinical investigation 2005 January; 115(1):187-94.
55. Kohler G., and C. Milstein. Continuous cultures of fused cells secreting antibody of predefined specificity. Nature 1975 Aug. 7; 256(5517):495-7.

What is claimed is:

1. A method of detecting at least one specific antibody to Sperm-Associated Antigen 16's (SPAG16's) presence and/or quantity, the method comprising:
contacting a body fluid from a mammal with SPAG16 protein and/or a peptide fragment of at least 5 consecutive amino acids of SPAG16, and
detecting the presence or measuring the quantity of specific antibodies bound to SPAG16 protein and/or the peptide fragment.

2. The method according to claim 1, wherein the method utilizes a peptide fragment of SPAG16 corresponding to SEQ ID NO:8.

3. The method according to claim 1, further comprising:
detecting the presence or quantity of at least one antibody that has a specificity for a peptide comprising a peptide selected from the group consisting of SEQ ID NO:1, SEQ ID NO:2, SEQ ID NO:3, SEQ ID NO:4, SEQ ID NO:5, SEQ ID NO:6, SEQ ID NO:7, and a fragment comprising at least 5 consecutive amino acids of SEQ ID NO:1, SEQ ID NO:2, SEQ ID NO:3, SEQ ID NO:4, SEQ ID NO:5, SEQ ID NO:6, or SEQ ID NO:7.

4. The method according to claim 1, wherein the mammal is a human.

5. The method according to claim 1, wherein the body fluid comprises cerebrospinal fluid.

6. The method according to claim 1, wherein the body fluid comprises blood, blood serum, and/or blood plasma.

7. The method according to claim 1, wherein detecting comprises an immune-enzymatic process comprising an enzyme-linked immunosorbant assay (ELISA), an immunofluorescent technique, a radioimmunological assay (RIA), immunoblotting, and/or a LINE blot.

8. The method according to claim 1, wherein detecting and/or quantifying at least one specific antibody indicates multiple sclerosis in the mammal.

9. A method of diagnosing multiple sclerosis in a mammal, the method comprising:
contacting a body fluid of the mammal with Sperm-Associated Antigen 16 (SPAG16) protein or a peptide fragment of at least 5 consecutive amino acids of SPAG16 protein, and
detecting the presence or measuring the quantity of at least one specific antibody bound to SPAG16 protein or the peptide fragment,
wherein the presence of and/or increased quantity of bound antibody as compared to a negative control indicates multiple sclerosis in the mammal.

10. A method for evaluating the prognosis and/or disease severity of multiple sclerosis in a subject suffering therefrom, the method comprising:
contacting a body fluid of the subject with Sperm-Associated Antigen 16 (SPAG16) protein and/or a peptide fragment of at least 5 consecutive amino acids of SPAG16 protein, and
detecting the presence or measuring the quantity of at least one specific antibody bound to SPAG16 protein and/or the peptide fragment,
wherein the decreased or increased concentration of the at least one specific antibody indicates the prognosis of multiple sclerosis in the subject.

11. A method for selecting a patient for a specific therapeutic treatment of multiple sclerosis or evaluating the therapeutic treatment of multiple sclerosis in a subject suffering therefrom, the method comprising:
contacting a body fluid of the subject with Sperm-Associated Antigen 16 (SPAG16) protein or a peptide fragment of at least 5 consecutive amino acids of SPAG16 protein, and
detecting the presence or measuring the quantity of the specific antibodies bound to SPAG16 protein or the peptide fragment,
wherein the presence or quantity of the antibody leads to an election of a specific therapeutic treatment of multiple sclerosis in the subject.

12. A diagnostic kit comprising:
Sperm-Associated Antigen 16 (SPAG16) protein or a peptide fragment of at least 5 consecutive amino acids of SPAG16 protein,
reagents for making a medium appropriate for an immunological reaction to occur, and
reagents that detect an antigen/antibody complex that has been produced by the immunological reaction.

13. The kit of claim 12, comprising an antibody specific for SPAG16 and/or a peptide fragment of at least 5 consecutive amino acids of SPAG16 protein.

14. SPAG16 or a peptide fragment of at least 5 consecutive amino acids of SPAG16 protein bound to a solid support.

15. A peptide comprising SEQ ID NO:8 bound to a solid support or label.


SEQUENCE LISTING (16 sequences)

SEQ ID NO: 1 - protein, 12 aa, Homo sapiens
ASSRGYEDLRTF

SEQ ID NO: 2 - protein, 23 aa, Homo sapiens
LDNSYHDNPVVSKELRIEGNQLT

SEQ ID NO: 3 - protein, 53 aa, Homo sapiens
LRAPAGLGAACDNWCIWQEQQLPLGATAGAANQRNTDTPHRATASLGAWSPPR

SEQ ID NO: 4 - protein, 14 aa, Homo sapiens
GARCINAEQPCQSP

SEQ ID NO: 5 - protein, 11 aa, Homo sapiens
YSCLKLYSFAN

SEQ ID NO: 6 - protein, 104 aa, Homo sapiens [sequence omitted]

SEQ ID NO: 7 - protein, 55 aa, Homo sapiens
GTGSGQGEEAAYLTNQPLCGPPASWLGGRALNQQGPRREEEVGQSLASPLGSFAI

SEQ ID NO: 8 - protein, 121 aa, Homo sapiens
ADDNFSIPEGEEDLAKAIQMAQEQATDTEILERKTVLPSKHAVPEVIEDFLCNFLIKMGMTRTLDCFQSEWYELIQKGVTELRTVGNVPDVYTQIMLLENENKNLKKDLKHYKQAAEYVIF

SEQ ID NO: 9 - DNA, 603 bp, Homo sapiens
ggcaagttcaagaggatatgaagatttgagaactttttaactattcattgactaaaaatgaacattaatgttaaagacttaagactttaacctgctggcagtcccaaatgaaattatgcaactttgatatcatattccttgatttaaattggcttttgtgattgagtgaaactttataaagcatatggtcagttatttaattaaaaaggcaaaacctgaaccaccttctgcacttaaagaagtctaacagtacaaatacactatctatcttagatagatatatttttttttatttttaaatattgtactatttatggtggtggggctttcttactaatacacaaataaatttaatcatttcaaaggcattctatttggtttagaagttgattcccaggagtgccatatttcagctactgtatttcctttttcttgtaatgtaagcagctcagataccatgtgctatcatttttgtatcaagttttttgcacaggatgtgaccactgtcagatcactgttcttttctttctttttgtgattgaaaagcctatactacaatttgaagtaaatttttgtttttcttaaaaaaaaaaaaaaaaaaaa

SEQ ID NO: 10 - DNA, 783 bp, Homo sapiens
ccttgataacagctaccatgacaaccctgtggtttccaaggagctgagaatagaaggaaactagcttacatgagaacagactggcctgaggagcagcagttgctggtggctaatggtgtaacctgagatggccctctggtagacacaggatagataactctttggatagcatgtctttttttctgttaattagttgtgtactctggcctctgtcatatcttcacaatggtgctcatttcatgggggtattatccattcagtcatcgtaggtgatttgaaggtcttgatttgttttagaatgatgcacatttcatgtattccagtttgtttattacttatttggggttgcatcagaaatgtctggagaataattctttgattatgactgttttttaaactaggaaaattggacattaagcatcacaaatgatattaaaaattggctagttgaatctattgggattttctacaagtattctgcctttgcagaaacagatttggtgaatttgaatctcaatttgagtaatctgatcgttctttctagctaatggaaaatgattttacttagcaatgttatcttggtgtgttaagagttaggtttaacataaaggttattttctcctgatatagatcacataacagaatgcaccagtcatcagctattcagttggtaagcttccaggaaaaaggacaggcagaaagagtttgagacctgaatagctcccagatttcagtcttttcctgtttttgttaactttgggttaaaaaaaaaaaaaaaaaaaa

SEQ ID NO: 11 - DNA, 539 bp, Homo sapiens
aattctcagggcccccgcaggcctgggagcggcctgtgataactggtgtatctggcaggagtagcagctgccccttggcgcgactgctggagccgcgaactagagaaacacagacacgcctcatagagcaacggcgtctctcggagcgtggagcccgccaaggtaactccgggaattgagtggagtggaggctgcactgaggcccccttctggctcctctctggtcgaaaaggctcccccgctagagagagccctgctgcttttggaagccttcaggtgttagttgctttgcaccgaagctgctgactgctggggttctgcgccgattgccaggctcaagtttattttgggggctgctgagcagactttgcccttctgtgctgttatcagcttctgtcgctgtccgtccacgcccgctatatccatccactctccgttctgtctccagcaggcaactccccctaccctgccccattttctgggtcataggggtatttgaaataaacctttaagggaaaagcaaaaaaaaaaaaaaaaaa

SEQ ID NO: 12 - DNA, 1119 bp, Homo sapiens
ggtgcaagatgcataaacgcagaatagccatgctagagcccctaactgtagacctgaatccacagtattatctgttggtcaacagacagatccagtttgaaattgcacatgcttactatgatatgatggatttgaaggttgccattgctgacaggctaagggatcctgattcacacattgtaaaaaaaataaataatcttaataagtcagcactgaagtactaccagctcttcttagactccctgagagacccaaataaagtattccctgagcatataggggaagatgttcttcgccctgccatgttagctaagtttcgagttgcccgtctctatggcaaaatcattactgcagatcccaagaaagagctggaaaatttggcaacatcattggaacattacaaatttattgttgattactgtgaaaagcatcctgaggccgcccaggaaatagaagttgagctagaacttagtaaagagatggttagtcttctcccaacaaaaatggagagattcagaaccaagatggccctgacttaatccttgtttttaaagaaaggaaatgtgcaatattgaagtgatctttttccctagtcagacaggcccaattccattgtgatgtttacctttatagccaggtgagtgcagtttgaacttgagatacagtcaactgagtgtttgctaggatcctaaggaacataaagttaattaaaaacttacacctaattatgtaaattgccttgttaaagacatgtgatttgtattttagatgcttgtttcctattaaaatacagacatttctaccctcagtttctaaatgtagactatttgttggctagtacttgatagattccttgtaagaaaaaatgctgggtaatgtacctggtaacaagcctgttaatatattaagattgaaaaagtaacttctatagttactccttctaaaatatttgacttcctaaattccccccacccaaaatctttcccttttgaaaatactaaaaactaagttatgttattataaagtgtaaaatggtttgtcttaattataggagaaaaaggccttgttagaaataaaataaactgacttatttcactaatgaaaaaaaaaaaaaaaaaa

SEQ ID NO: 13 - DNA, 864 bp, Homo sapiens
ctattcttgcttaaaactgtactcttttgcaaattaacaattttatcactgattcagagttaaaaagaagactaacttttcaagcaaatgcatctgtaaagatgctttagattagactgtcatgtctcagtgtctatctgtatatattatttgatattcagagaatctaaagcactcgtctactgttttaatgagatttaacagcttttaacagtgagtttcgtttgtaaactgcttgaagtctgtggcattcaggcacacgtctggctggccggctgggtctcctcccgggctcagtgggcctggggcctctctgacgtggtgcctgctggagggaggctcgtcgtcaccagctgactgctggtccggcttctgaccggcctttgtcctggctccgtagcagaacactgtaaaagtgcccgcgtctttgcagtagttgcagatttcagtcgtcgtgttacttgtgcacaaacagaagctgggtcttacccgcagcacgagtgtctcgggctgcccggagtcgcccgggagcaggtgctgcagccagagttacgcgggggccacgcgggccggcgggggtggggggaacgtgggggaacctgtgtttcacgtgactcagcagtgcccgccgccgtcaccagctatgcattcactccgtttccagtgagcagatgtcttgcttggaaagtggacctgtgtctgtgtctgtcctgagaacttaccagcagaaatcctcatttctgtgctacggatttaccaaaaattgtcaagtctttttcagtttaacagttcctttacatgtgtagtatttgaggaaaaaaatcaataaacagttgatctcgtgcataaaaaaaaaaaaaaaaa

SEQ ID NO: 14 - DNA, 790 bp, Homo sapiens
aacatgctacacaaaaccaggtttctgtttcagataaataggtgaagggactcttaatcctaaagactgaaaagtagaaaagaaaggggaagaagagtgcctcaaggattgccattggaggttcttttgctggggctgattgccagctgagattattcaagccccagagcaaacattctgctcctgctcccttagagctgccctcccaccgctcagtattgcctcctgcgaggggcgggctggctgccgcagacaccagtgaaccctttttccattccagaagtcccagtggacctactttaatataccaataacactcctattttaaactagctgtatccattttcgttttaatagtcccagtgctaaagtttttcaaagcagttattttgtaagtaggtcaaacaggtactttgggatcctgttctgtctgtttgcttgccaggtaacctctttgttatctaattcaaagtctggtacagtttgaaccaaaacaaaaaaggaatgatgtttcactttggagtcaagattcattcattttctaacattaatcattttcgttatacagtaagtctatattcatgataaaaaatagaaaatatgaataagcaaaactaaattgaaaggaaaaccatctgtgatctgccaattagaaaatctctattctaaacattttggtaaatatgctaccagatttttatctatgcaaatgtgtatctgtatttttcctcacttgtatagtggacatcttttcatattaataaataagttagcatcaaaaaaaaaaaaa

SEQ ID NO: 15 - DNA, 681 bp, Homo sapiens
ggacagggagtgggcaaggggaagaagcagcttatttgactaaccagcccctctgtggtccaccagcgtcttggcttggtgggagggctctcaatcagcagggccccaggagggaagaagaagtggggcaaagcctggcctcgccgctcgggagctttgccatctgagccacgcctcctccaggccatgctccttgaacttggaaatgtcaaccggagcccttacaccagccctccagcatctaatagacttgaatctactctaaacgaatatttaatccaacctcactacattgtagctcagtccaacgactaaccctgaaatgggggtgttccagccttcagcgagatggccaagcggtcccctgggggctgtggcagcgggcttatccttctctgttgccaaccttgccgtccgacctcctccgcccccatgcggtgaccccgtccgtgtctgtgtctgtccatacgtgtgagtccagctaaaaagacaaaacagaacccgtgggcccagctcggaaggtgcgtggagaaggctccgacgtctccgaagtgcagcccttgggatggcattccgttgtgtgccttattcctggagaatctgtatacggctcgcctatagaaatatagcctcttcatgctgtattaaaaggacttttaaaagcaaaaaaaaaaaaaaaaa

SEQ ID NO: 16 - DNA, 970 bp, Homo sapiens
cagatgacaattttagcatcccagaaggtgaagaagatctggcaaaagcaattcagatggcccaagaacaggctacagatactgaaattttggaacggaaaacagttcttccttcaaagcatgcagtacctgaagtaatagaagactttctctgcaatttcttgatcaaaatgggaatgaccagaactcttgattgctttcagtctgaatggtatgagttaatacagaaaggagtgactgaacttagaactgttgggaatgttccagatgtctacacccagattatgctgttggaaaatgagaacaaaaatttaaagaaagatttgaagcactacaaacaagcagctgagtatgttattttttaaatgacattttcttctttttcttttggactaaataaaagagttgagtgaagctgatatatgtaatataccagagccttaatttttgaaaactgaatttttctagttgtaaagaatgtgagaggcttcattagcaaattaattaaacagatgatcagaactatcacaattataacttaccaacaagaagggaatgcaggtagttgtttaggagatggtacattttttatataacattcacttccttgtgtatttgatagtcttttcatggtttataacattttctcctgtaaagataggctaatttctgaaataataattaaatttatagaaagccgagaggaaattgctagtttattcctggtagaggaatttctgtatttgaaaattctccagaaggaataatataaactgtggactttgggtgataatgatatgtaggttcgtcagttgttaacaaatgtatccctctgttgggggctattgataatggggaaggctgtgcatgtgtgggagtaggaggtgtatgggacatctctgtaccttctaatcaattttgctatgaacttaaaactgctctaaaaataaaaaaaaaaaaaaaaaaaaaa